(19) United States
(12) Patent Application Publication
(10) Pub. No.: US 2016/0113732 A1
(43) Pub. Date: Apr. 28, 2016

(54) SURGICAL TOOL

(71) Applicant: AGILE ENDOSURGERY, INC., Chapel Hill, NC (US)

(72) Inventors: Adam T.C. STEEGE, Chapel Hill, NC (US); Theodore J. MOSLER, Raleigh, NC (US)

(21) Appl. No.: 14/897,043

(22) PCT Filed: Jun. 10, 2014
(86) PCT No.: PCT/US2014/041722

§ 371 Date: Dec. 9, 2015

Related U.S. Application Data
(60) Provisional application No. 61/833,251, filed on Jun. 10, 2013.

(51) Int. Cl. A61B 34/00; A61B 17/29

(52) U.S. Cl. CPC A61B 34/71; A61B 17/29; A61B 2017/00367

(57) ABSTRACT

A surgical tool comprises a manipulator adapted to receive at least a portion of a hand of an operator. A proximal universal joint has a first end mounted to the manipulator. A hollow elongated member has a first end mounted to a second end of the proximal universal joint. A distal universal joint has a first end mounted to a second end of the elongated member. An end effector comprises a universal joint element pivotally mounted to a second end of the distal universal joint for rotation about a first axis, and a base member pivotally connected to the joint element for rotation about a second axis perpendicular to the first axis. Pivoting of the first end of the proximal universal joint causes the end effector to move in a corresponding motion.

### CROSS REFERENCE TO RELATED APPLICATION

This application claims the benefit of U.S. Provisional Application No. 61/833,251, filed Jun. 10, 2013, entitled “SURGICAL TOOL,” the contents of which are hereby incorporated by reference in their entirety.

### BACKGROUND

Embodiments described herein generally relate to surgical apparatus for tissue and suture manipulation, and more particularly to apparatus that may be applied to conducting laparoscopic and endoscopic surgery.

Minimally invasive (endoscopic) surgery encompasses a set of techniques and tools, which are becoming more and more commonplace in the modern operating room. Minimally invasive surgery causes less trauma to the patient when compared to the equivalent invasive procedure. Hospitalization time, scarring, and pain are also decreased, while recovery rate is increased.

Endoscopic surgery is accomplished by the insertion of a trocar containing a cannula to allow passage of endoscopic tools. Optics for imaging the interior of the patient, as well as fiber optics for illumination and an array of grasping and cutting devices are inserted through a multiple cannulae, each with its own port.

Currently the majority of cutting and grasping tools are essentially the same in their basic structure. Standard devices consist of a user interface at the proximal end and an end effector at the distal end of the tool used to manipulate tissue and sutures. Connecting these two ends is a tube section, containing cables or rods used for transmitting motion from the user interface at the proximal end of the tool to the end effector at the distal end of the tool. The standard minimally invasive devices (MIDs) provide limited freedom of movement to the surgeon. The cannula has some flexibility of movement at the tissue wall, and the tool can rotate within the cannula, but tools cannot articulate within the patient's body, limiting their ability to reach around or behind organs or other large objects. Several manually operated devices have attempted to solve this problem with articulated surgical tools that are constructed much in the same way as standard MIDs. These devices have convoluted interfaces, making them more difficult to control than their robotic counterparts. Many lack torsional rigidity, limiting their ability to manipulate sutures and denser tissue.

Robotic surgical instruments have attempted to solve the problems that arise from the limitations of standard MIDs with telemetrically controlled articulated surgical tools. However, these tools are often prohibitively expensive to purchase and operate. The complexity of the devices raises the cost of purchasing as well as the cost of a service contract. These robotic solutions also have several other disadvantages such as complications during the suturing process. An additional and critical disadvantage is their lack of haptic feedback, which has been known to lead to serious complications.

In the case of both articulated hand-held devices and robotic devices, the issue of compactness and strength are high priorities in terms of design. Many previously proposed articulated devices require a significant amount of space to articulate properly. Furthermore, many previous articulated instruments present too large of a cost burden to be widely adopted by smaller hospitals. Instruments that are partially disposable and partially reusable (reposable) have been developed to address this issue.

### SUMMARY OF THE INVENTION

Embodiments of a surgical instrument are disclosed for use in a wide variety of roles including grasping, dissecting, clamping, or retracting materials or tissue during surgical procedures performed within a patient's body and particularly within the abdominal cavity.

The surgical instrument disclosed herein may include a handle portion, a proximal joint, an endoscopic tube portion, a distal joint, and a pair of jaws. The joint in one embodiment is controlled by four cables, which in turn also control the jaws. There are three primary motions that these cables actuate: rotation about a primary joint axis, rotation about a secondary joint axis, and the opening and closing of the jaws. The embodiment described below is such that the end effector may be controlled by a manual interface or a robotic interface.

The instrument described below is one embodiment that can control the joint and jaws manually. The four cables that control the distal joint and jaws pass through the endoscopic tube section to the proximal joint. This interface is similar to existing interfaces on endoscopic instruments, enabling comfortable use for any surgeon with prior endoscopic experience.

The jaws may be of any of a variety of configurations. They may be tailored to a specific task, such as suture grasping, tissue grasping, tissue dissection or electrocautery. The embodiment described below is such that all of these specific tasks can be easily adapted to the current description. Additionally, the present embodiment contains a force amplification mechanism which provides greater grip strength in the end effector. This is particularly suited for suture grasping, where the requisite grip forces are higher than for tissue manipulation. This mechanism is also suited for electrocautery and other applications where the requisite grip force is higher than may be readily achievable without the amplification mechanism.

Further, in one aspect an endoscopic surgical grasper is provided with a joint such that the grasper can articulate with two degrees of freedom.

In another aspect, the surgical grasper may be controlled robotically.

In another aspect, the surgical grasper may be controlled by a manual interface.

In another aspect, an endoscopic surgical end effector is provided that is adaptable to multiple different jaw structures for different surgical procedures.

In another aspect, an endoscopic surgical instrument is provided that utilizes a proximal joint and interface to control a distal joint and jaws for performing a variety of surgical tasks.

In another aspect, the aforementioned grasper is provided that contains a force amplification mechanism.

In another aspect, an endoscopic surgical instrument is provided that has detachable disposable components, while other components are reusable.

A surgical tool for use by an operator, comprises a manipulator adapted to receive at least a portion of a hand of the operator, a proximal universal joint having a first end and a second end, the first end of the proximal universal joint being mounted to the manipulator, a hollow elongated member having a first end, a second end, and a longitudinal axis, the first end of the elongated member being mounted to the second end of the proximal universal joint, a distal universal joint having a first end and a second end, the first end of the distal universal joint being mounted to the second end of the elongated member, and an end effector. The end effector comprises a universal joint element pivotally mounted to the second end of the distal universal joint for rotation about a first axis, and a base member pivotally connected to the joint element for rotation about a second axis perpendicular to the first axis. Pivoting of the first end of the proximal universal joint causes the end effector to move in a corresponding motion.

In one aspect, cabling operatively couples the proximal joint and the end effector, wherein the cabling comprises four cables that each engage the end effector.

The end effector may further comprise a digit pivotally mounted to the base member for movement relative to the base member between a closed position contacting the base member and an open position spaced from the base member. In this aspect, the end effector further comprises a driver pivotally mounted to the base member for movement relative to the base member, the driver including a cam element, wherein the digit defines an elongated opening for receiving the cam element such that the digit is movable between the closed position and the open position by movement of the driver relative to the base member. The opening in the digit may be arcuate for varying the force at different relative positions of the digit and the driver. In another aspect, cabling operatively couples the proximal joint and the driver, wherein the cabling comprises four cables that each engage the end effector.

In another aspect, the manipulator comprises an actuator operable to control the end effector. The actuator comprises a trigger assembly adapted to be operable with a finger of the operator, wherein actuating the trigger assembly causes the driver to move relative to the base member.

In a further aspect, the proximal and end effector universal joints each comprise a proximal end member and distal end member, with each end member including a base portion and opposing arms extending from the base portion, the proximal end member and the distal end member mounted to a center block for each joint, the center block pivotable around two substantially coplanar, perpendicular axes, wherein the base portions and center block define openings for receiving the end effector control cables. The proximal and end effector universal joints may be controlled by universal joint control cables anchored in the manipulator and may which be adjusted with means for tensioning the universal joint control cables.

Still further, the manipulator has a longitudinal axis and a first angular position, and the end effector has a longitudinal axis parallel to the longitudinal axis of the manipulator and a first angular position, and at all relative positions of the manipulator and the end effector, the longitudinal axis of the manipulator and the longitudinal axis of the end effector remain parallel, and the degree of rotation of the manipulator about the longitudinal axis of the manipulator from the first angular position of the manipulator is equal to the degree of rotation of the end effector about the longitudinal axis of the end effector from the first angular position of the end effector.

An articulation system is provided for a surgical tool. The articulation system comprises a proximal universal joint including a proximal end member and a distal end member, a hollow elongated member having a first end, a second end, and a longitudinal axis, the first end of the elongated member first end being mounted to the distal end member of the proximal universal joint, an end effector universal joint comprising a proximal end member and a distal end member, the proximal end member of the end effector universal joint being mounted to the second end of the elongated member, and universal joint control cables operatively connecting the proximal universal joint and the end effector universal joint, wherein pivoting motion of the proximal end member of the proximal universal joint relative to the longitudinal axis of the elongated member exerts force on the control cables to cause a corresponding pivoting motion of the distal end member of the end effector universal joint.

In one aspect, the articulation system comprises a base control element rigidly mounted to the proximal end member of the proximal universal joint, and a cable tensioner pivotally mounted to the base control element, wherein the control cables are operatively connected to the tensioner.

In another aspect, the articulation system further comprises a brake assembly for engaging the proximal joint.

In a further aspect, the articulation system further comprises a detensioner including a base member operatively connected to the first end of the elongated member, a slide member operatively mounted to the base member for linear movement relative to the base member between a first position adjacent the base member and a second position spaced from the base member, the slide member engaging the proximal joint, and an arm pivotally connected to the base member for rotation in a plane parallel to the direction of linear movement of the slide member, and means for biasing the slide member to the first position, wherein rotation of the arm engages and moves the slide member from the first position to the second position.

In the articulation system, each end member of the proximal universal joint and the end effector universal joint may include a base portion and opposing arms extending from the base portion, wherein each respective proximal end member and distal end member are mounted to a center member at the arms of the proximal end member and the distal end member, and wherein the center members permit pivoting of the proximal and distal end members around two substantially coplanar, perpendicular axes through the center member. Each of the proximal universal joint and the end effector universal joint may include round elements interposed between the center member and the arms at the mounting locations of the end members to the center member, and which may be independent parts or integral to the center member or arms, and wherein the round elements are engaged by the universal joint control cables. Four universal joint control cables operatively connect the proximal universal joint and the end effector universal joint.

In yet another aspect of the articulation system, the proximal end member of the proximal universal joint has a longitudinal axis and a first angular position, and the distal end member of the end effector has a longitudinal axis and a first angular position, and wherein at all relative positions of the proximal end member of the proximal universal joint and the distal end member of the end effector universal joint, the longitudinal axis of the proximal end member of the proximal universal joint and the longitudinal axis of the distal end member of the end effector remain parallel, and the degree of rotation of the proximal end member of the proximal universal joint about the longitudinal axis of the proximal end member of the proximal universal joint from the first angular position of the proximal end member of the proximal universal joint is equal to the degree of rotation of the distal end member of the end effector universal joint about the longitudinal axis of the distal end member of the end effector from the first angular position of the end effector universal joint.

Further features of the subject invention will become more readily apparent from the following detailed description of the invention taken in conjunction with the accompanying drawings.

### BRIEF DESCRIPTION OF THE DRAWINGS

Preferred embodiments of the subject invention will be described herein below with reference to the drawings, wherein

FIG. 1 is a left perspective view of one embodiment of the subject invention.

FIG. 2 is a left view of the surgical instrument in FIG. 1 in an articulated configuration about a first axis of rotation.

FIG. 3 is a top view of the surgical instrument in FIG. 1 in an articulated configuration about a second axis of rotation.

FIG. 4 is a left perspective exploded view of the surgical instrument in FIG. 1 with reusable and disposable components separated.

FIG. 5 is a left perspective view of the cartridge assembly of the surgical instrument in FIG. 1, this assembly containing the disposable components of the subject invention.

FIG. 6 is a left perspective exploded view of the cartridge assembly in FIG. 5.

FIG. 7 is a left perspective view of the end effector assembly of the cartridge assembly in FIG. 5.

FIG. 8 is a left perspective exploded view of the end effector assembly shown in FIG. 7.

FIG. 9 is a left perspective sectional view from above of the cabled end effector assembly in FIG. 7.

FIG. 10 is a left perspective sectional view from below of the cabled end effector assembly in FIG. 7.

FIG. 11 is a right perspective sectional view from above of the cabled end effector assembly in FIG. 7.

FIG. 12 is a right perspective sectional view from below of the cabled end effector assembly in FIG. 7.

FIG. 13 is a left perspective sectional view from above of the cabled end effector assembly in FIG. 7 in a configuration with the jaw element in a proximately open position.

FIG. 14 is a left perspective sectional view from above of the cabled end effector assembly in FIG. 7 in a configuration with the jaw element in a partially closed position.

FIG. 15 is a left perspective view from above of the cabled end effector assembly in FIG. 7 in an articulated configuration with respect to a first axis of articulation.

FIG. 16 is a sectional view of the end effector in FIG. 15.

FIG. 17 is a left perspective view from above of the cabled end effector assembly in FIG. 7 in an articulated configuration with respect to a second axis of articulation.

FIG. 18 is a sectional view of the end effector in FIG. 17.

FIG. 19 is a left perspective view from above of the proximal control assembly of the cartridge assembly in FIG. 5.

FIG. 20 is an exploded view of the proximal control assembly in FIG. 19.

FIG. 21 is a left perspective sectional view from above of the cabled proximal control assembly in FIG. 19.

FIG. 22 is a left perspective sectional view from below of the cabled proximal control assembly in FIG. 19.

FIG. 23 is a right perspective sectional view from above of the cabled proximal control assembly in FIG. 19.

FIG. 24 is a right perspective sectional view from below of the cabled proximal control assembly in FIG. 19.

FIG. 25 is a left perspective view from above of the proximal control assembly in FIG. 19 in a position corresponding to the proximately open jaw position in FIG. 13.

FIG. 26 is a left perspective sectional view from above of the proximal control assembly in FIG. 25.

FIG. 27 is a left perspective sectional view from below of the proximal control assembly in FIG. 25.

FIG. 28 is a left perspective view from above of the proximal control assembly in FIG. 19 in an articulated configuration with respect to a first axis of articulation.

FIG. 29 is a left perspective sectional view from above of the proximal control assembly in FIG. 29.

FIG. 30 is a left perspective sectional view from below of the proximal control assembly in FIG. 29.

FIG. 31 is a left perspective view from above of the proximal control assembly in FIG. 19 in an articulated configuration with respect to a second axis of articulation.

FIG. 32 is a left perspective sectional view from above of the proximal control assembly in FIG. 31.

FIG. 33 is a left perspective sectional view from below of the proximal control assembly in FIG. 31.

FIG. 34 is a left perspective view from above of a schematic representation of the interaction between the distal end effector and the proximal control assemblies.

FIG. 35 is a left perspective view from above of the assemblies in FIG. 34 in a proximately open configuration.

FIG. 36 is a left perspective view from above of the assemblies in FIG. 34 in an articulated configuration with respect to their first axes of rotation.

FIG. 37 is a left perspective view from above of the assemblies in FIG. 34 in an articulated configuration with respect to their second axes of rotation.

FIG. 38 is a left perspective view from above of the proximal portion of the instrument shown in FIG. 1.

FIG. 39 is an exploded view of the instrument shown in FIG. 38.

FIG. 40 is a left sectional view of the instrument shown in FIG. 38 with the trigger element in a proximately open position.

FIG. 41 is a left perspective sectional view from above of the instrument shown in FIG. 38 with the trigger element in a partially closed position.

FIG. 42 is a left sectional view of the instrument shown in FIG. 38 with the trigger element in a proximately closed position.

FIG. 43 is a left sectional view of the instrument shown in FIG. 42 with the trigger latch release element in a proximately engaged position.

FIG. 44 is a left perspective view from above of the trigger clutch element of the instrument shown in FIG. 38.

FIG. 45 is a right perspective view from above of the trigger clutch element of the instrument shown in FIG. 38.

FIG. 46 is a left perspective view from above of the trigger element of the instrument shown in FIG. 38.

FIG. 47 is a back perspective view from above of the trigger element in FIG. 46.

FIG. 48 is a left perspective view from above of the brake cam control element of the instrument shown in FIG. 38.

FIG. 49 is a front view of the brake cam control element shown in FIG. 48.

FIG. 50 is a left perspective view from above of the brake cam element of the instrument shown in FIG. 38.

FIG. 51 is a left view of the brake cam element shown in FIG. 50.

FIG. 52 is a left perspective view from above of the brake actuation element of the instrument shown in FIG. 38.

FIG. 53 is a left view of the brake actuation element shown in FIG. 52.

FIG. 54 is a left perspective view from above of the brake assembly of the instrument shown in FIG. 38.

FIG. 55 is an exploded view of the brake assembly shown in FIG. 54.

FIG. 56 is a left sectional view of the brake assembly shown in FIG. 54.

FIG. 57 is a left perspective view of the first ratchet element of the instrument shown in FIG. 38.

FIG. 58 is a left perspective view of the trigger latch element of the instrument shown in FIG. 38.

FIG. 59 is a second perspective view of the trigger latch element shown in FIG. 58.

FIG. 60 is a left perspective view from below of the trigger latch release element of the instrument shown in FIG. 38.

FIG. 61 is a left perspective view from above of the trigger latch release element of the instrument shown in FIG. 38.

FIG. 62 is a left perspective view from above of the detensioning assembly of the instrument shown in FIG. 1 in a proximately compressed configuration.

FIG. 63 is a left perspective view from above of the detensioning assembly of the instrument shown in FIG. 1 in a proximately expanded configuration.

FIG. 64 is an exploded view of the detensioning assembly shown in FIG. 62.

FIG. 65 is a left sectional view of the detensioning assembly shown in FIG. 62.

FIG. 66 is a left sectional view of the detensioning assembly shown in FIG. 63.

FIG. 67 is a left perspective view from above of the primary base element of the detensioning assembly shown in FIG. 62.

FIG. 68 is a right perspective view from above of the primary base element of the detensioning assembly shown in FIG. 62.

FIG. 69 is a left perspective view from above of the secondary base element of the detensioning assembly shown in FIG. 62.

FIG. 70 is a right perspective view from above of the secondary base element of the detensioning assembly shown in FIG. 62.

FIG. 71 is a left perspective view from above of the arm element of the detensioning assembly shown in FIG. 62.

FIG. 72 is a left perspective view from below of the arm element of the detensioning assembly shown in FIG. 62.

### DETAILED DESCRIPTION

The components of the present embodiment of the subject invention are largely symmetric about the vertical plane. Terms such as “right,” “left,” “front,” and “back,” are given from the perspective of an individual using the instrument and are intended as a means for easier comprehension of the design and not to constrain the design. The majority of views are given from a left perspective, due to the symmetry of many of the components and assemblies. Features of asymmetric components are clarified with further views.

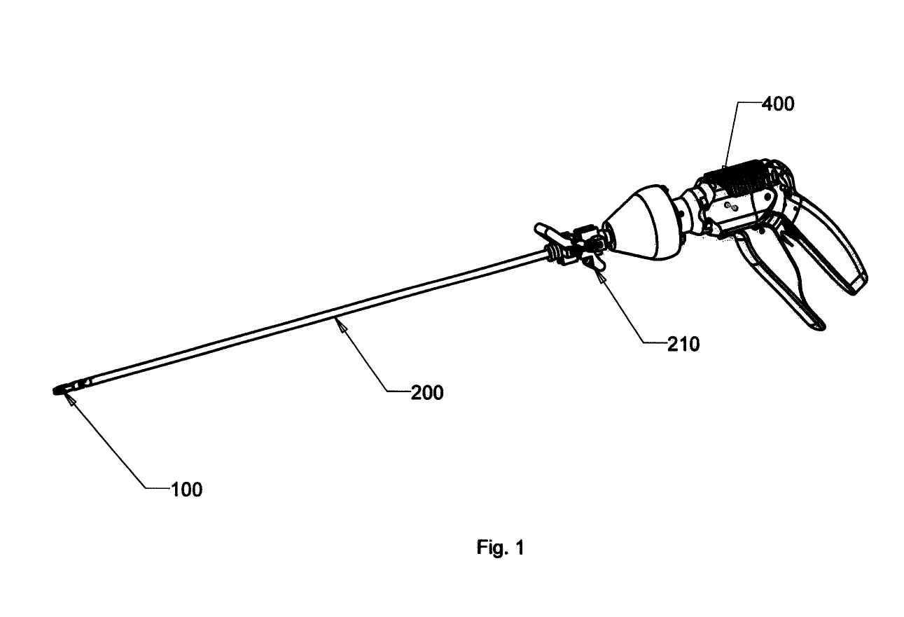

FIGS. 1-4 depict the structure and connection of the cartridge assembly (200) and the handle assembly (400) as well as the cartridge subassemblies such as the end effector assembly (100), tube portion (202), detensioner assembly (210), proximal joint (300), and proximal control assembly (350). The end effector assembly (100) is mounted to the end of the tube portion (202). The tube (202) is in turn mounted to the detensioner assembly (210) which connects to the proximal joint (300) and proximal control assembly (350). The cartridge assembly (200) is detachably connected to the handle (400).

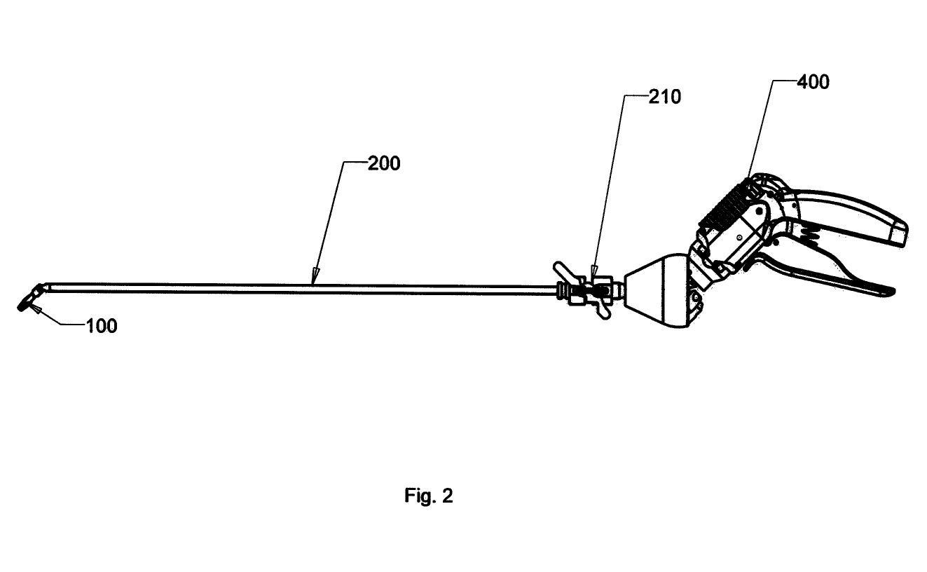

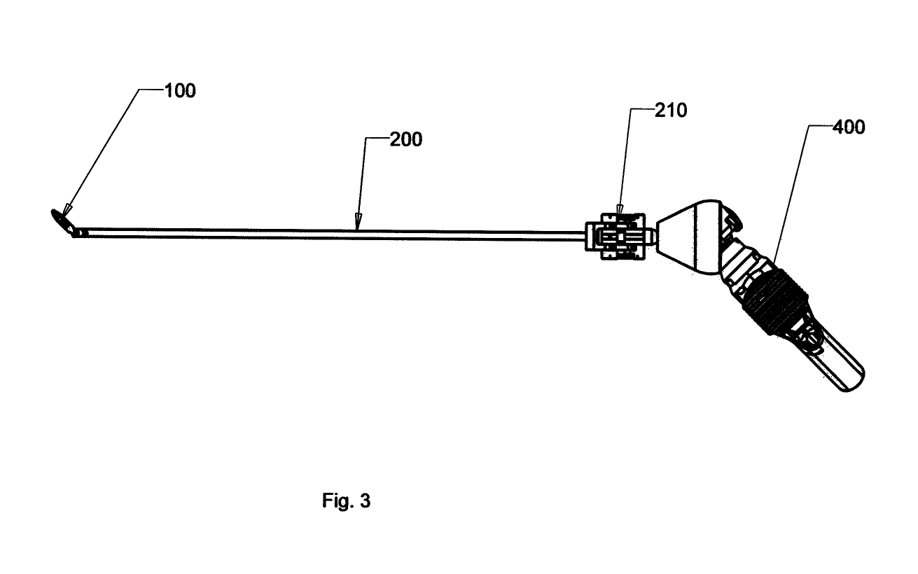

FIGS. 2 and 3 illustrate the functionality of the motion control system. When the handle (400) rotates in a counterclockwise direction as viewed from the left about a primary control axis as seen in FIG. 2, the end effector assembly (100) rotates similarly about a corresponding control axis such that the end effector assembly (100) remains aligned with the handle (400). When the handle (400) rotates in a clockwise direction as viewed from the top about a secondary control axis as seen in FIG. 3, the end effector assembly (100) rotates similarly about a corresponding control axis to produce the same effect. The combination of these two axial responses maintains alignment between the end effector assembly (100) and handle (400). The details of how this is achieved, as well as the means by which the cartridge assembly (200) is detachably connected to the handle (400) will be further specified.

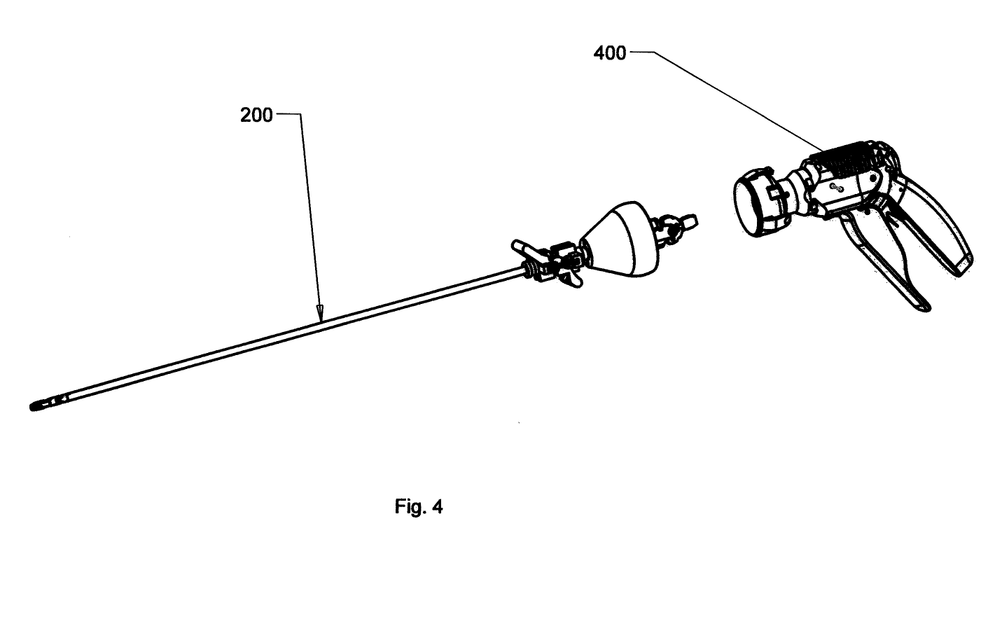

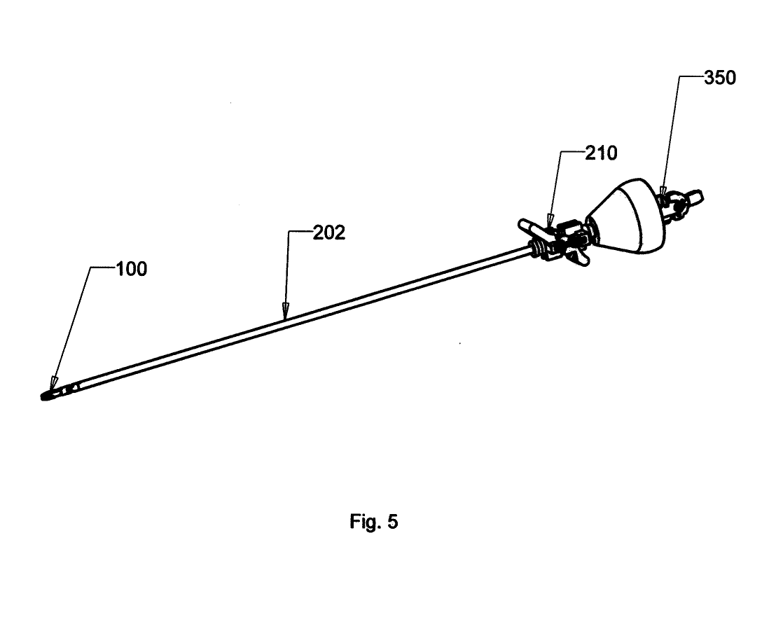

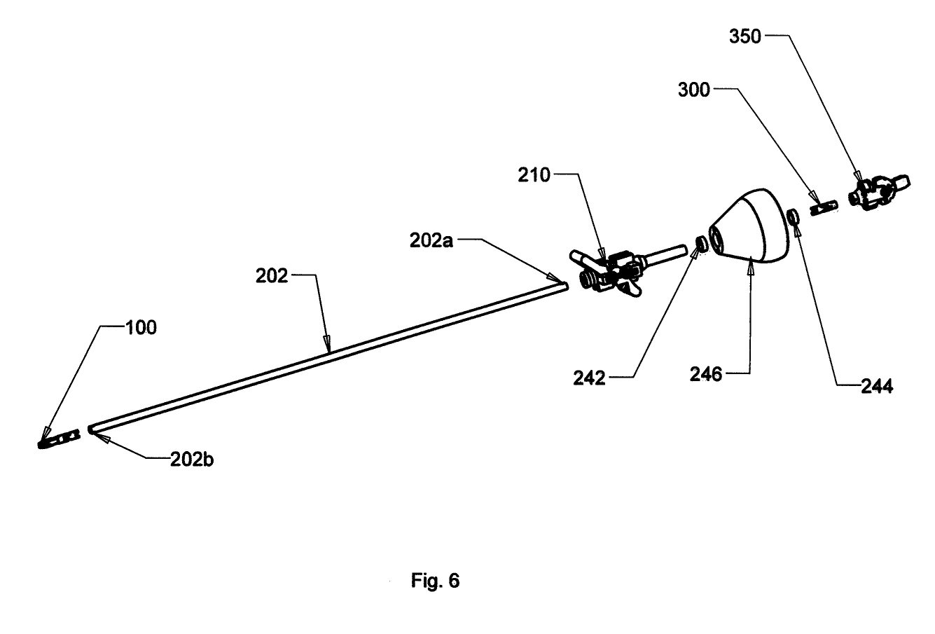

FIGS. 5 and 6 detail the components of the cartridge assembly (200). The end effector assembly (100) is controlled by the proximal joint (300) and proximal control assembly (350). The proximal joint block (246) constrains the angle through which the proximal joint (300) can articulate and provides means for locking the proximal joint (300) in a particular position; this feature will be further detailed below.

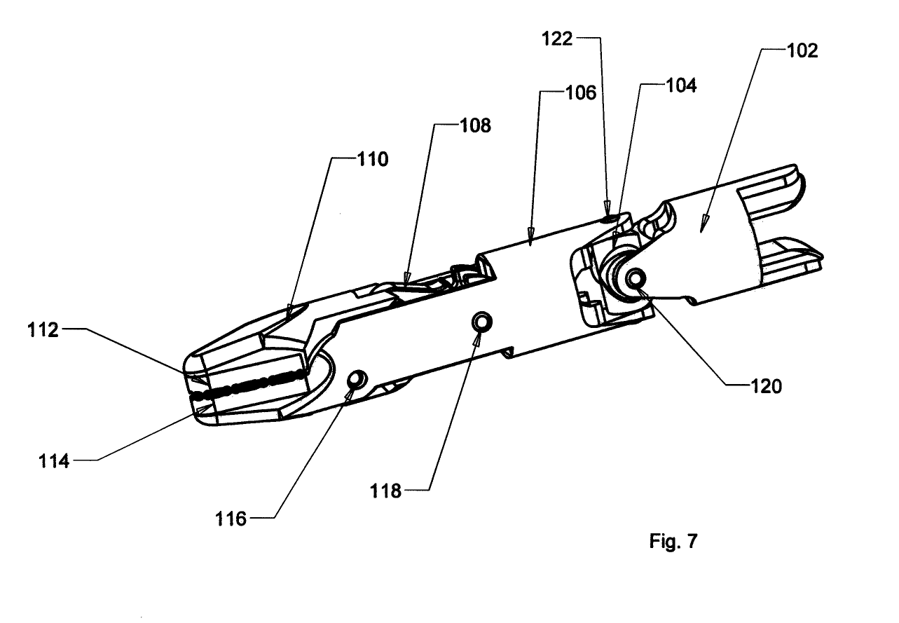

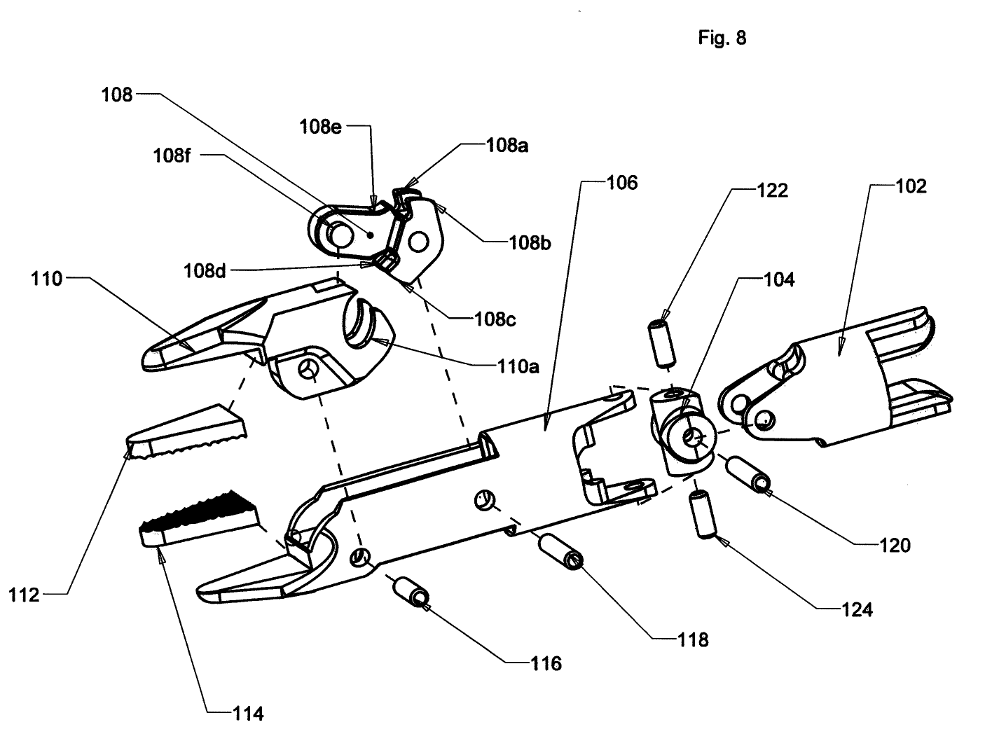

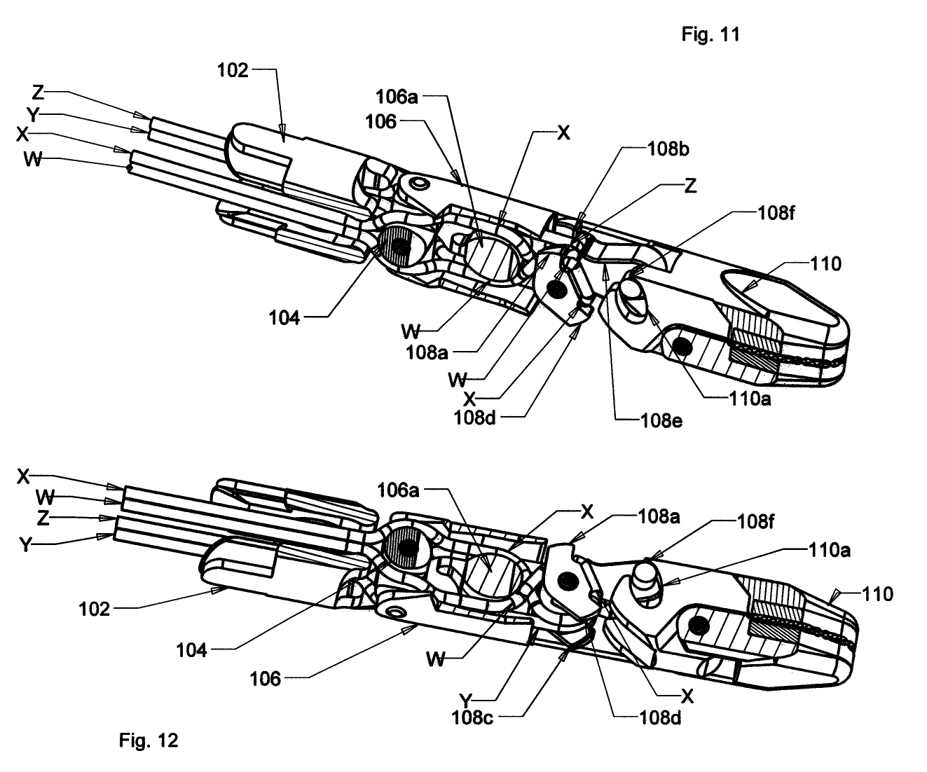

FIGS. 7-12 detail the components of the end effector assembly (100) and the cabling that controls it. The distal center joint element (104) is pivotally connected to the distal joint base element (102) via a pin (120) which defines a primary articulation axis. The jaw base (106) is pivotally connected to the distal center joint element (104) via two pins (122,124) which define a secondary articulation axis. These pivotal connections may in general be made by any number of pins or pin features which define two perpendicular axes of articulation. The jaw driver (108) is pivotally connected to the jaw base (106) by the jaw driver pin (118). The jaw (110) is pivotally connected to the jaw base (106) by the jaw pin (116). The jaw (110) and jaw base (106) contain grip inserts (112,114) which may be customized to the purpose of a particular instance of the subject invention. For example, a version of this device that is specifically for grasping sutures would likely have inserts made of a hard material with sharp teeth, whereas a version designed for grasping tissue would likely have inserts of softer, pliable material with rounded features.

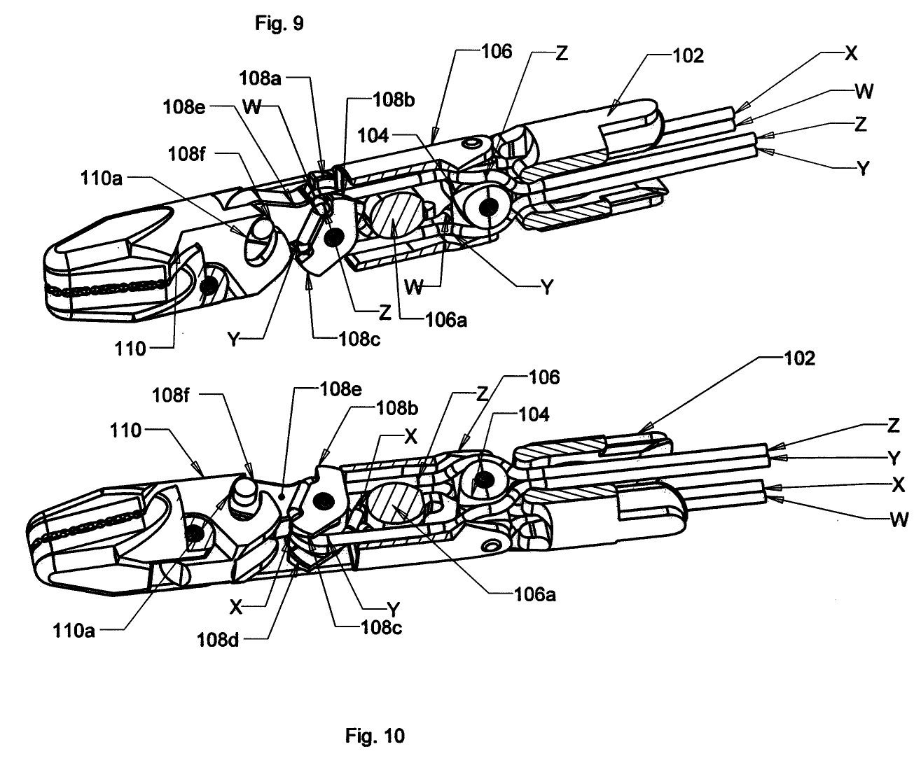

With specific reference to FIGS. 9-12, the control system for the end effector assembly (100) is described herein. There are four control cables (W,X,Y,Z) that produce three principal motions of the end effector assembly (100). These motions are articulation about a primary axis, articulation about a secondary axis, and actuation of the jaw (110). Cable W enters the distal joint base element (102) and passes underneath a horizontal round feature of the distal center joint element (104) before passing around the right side of a vertical round feature of the distal center joint element (104) and entering the jaw base (106). Cable X enters the distal joint base element (102) and passes over a horizontal round feature of the distal center joint element (104) before passing around the right side of a vertical round feature of the distal center joint element (104) and entering the jaw base (106). Cable Y enters the distal joint base element (102) and passes underneath a horizontal round feature of the distal center joint element (104) before passing around the left side of a vertical round feature of the distal center joint element (104) and entering the jaw base (106). Cable Z enters the distal joint base element (102) and passes over a horizontal round feature of the distal center joint element (104) before passing around the left side of a vertical round feature of the distal center joint element (104) and entering the jaw base (106). Cable W passes underneath the jaw base central guiding feature (106a) and over the top of the jaw driver (108) before being fixed in place by a cable retention feature (108a). Cable X passes over the jaw base central guiding feature (106a) and under the jaw driver (108) before being fixed in place by a cable retention feature (108d). Cables W and X are actually one continuous cable in the depicted instance of the subject invention; they are described as separate cables because they function equivalently to two separate cables fixed at the jaw driver (108). Cable Y passes underneath the jaw base central guiding feature (106a) and under the jaw driver (108) before being fixed in place by a cable retention feature (108c). Cable Z passes over the jaw base central guiding feature (106a) and over the jaw driver (108) before being fixed in place by a cable retention feature (108b). Cables Y and Z are actually one continuous cable in the depicted instance of the subject invention; they are described as separate cables because they function equivalently to two separate cables fixed at the jaw driver (108). In each of the above cable configurations, the tension of the cables locks the cables between the cable retention features (108a,108b,108c,108d) and the central body of the jaw driver (108e). In general, this cable fixation may be achieved by swaging, adhesive attachment, or any other method which fixes multiple cables to the cable driven element: in this instance, the jaw driver.

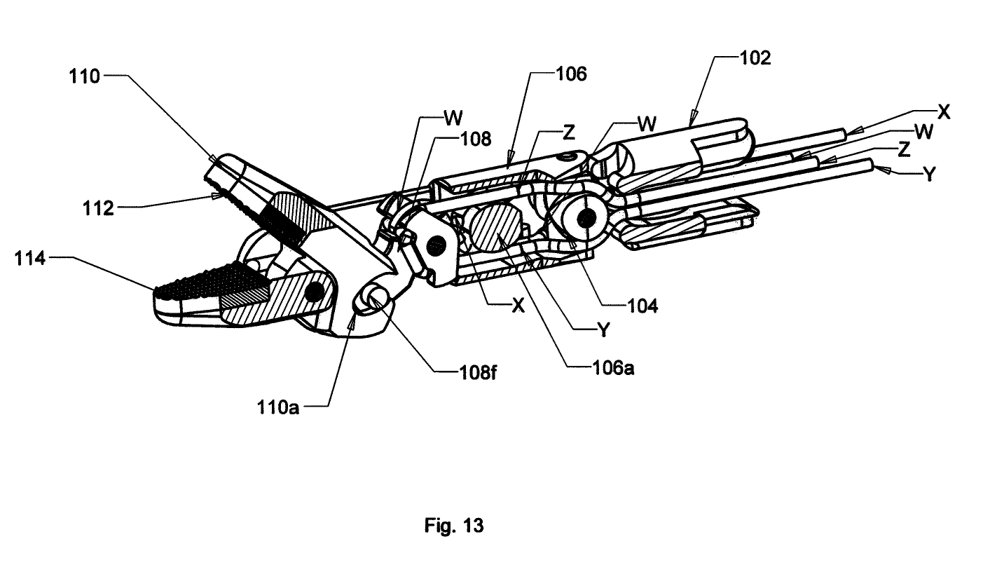

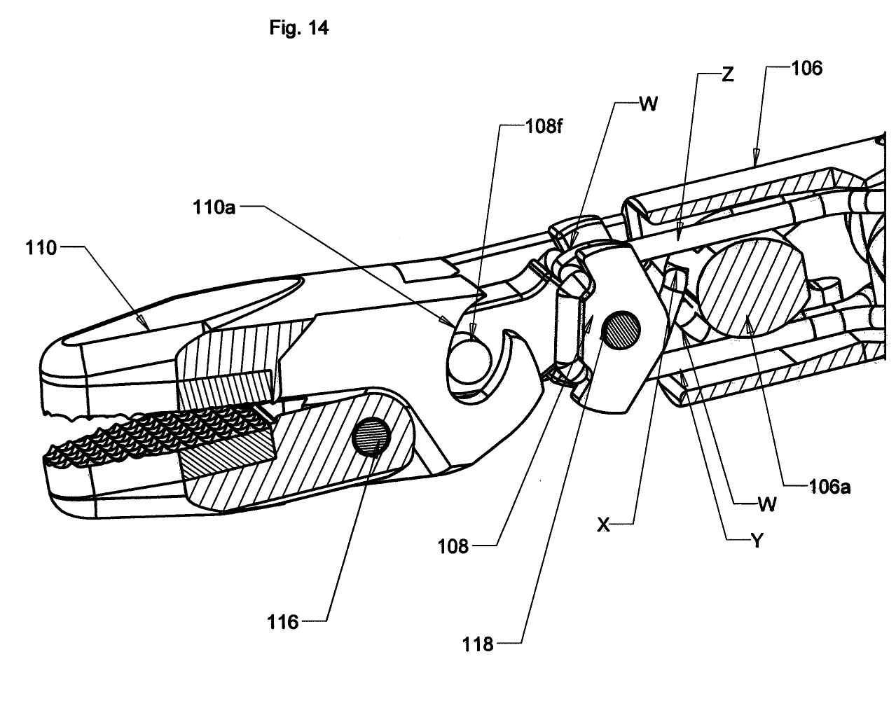

FIGS. 13 and 14 show the interaction between the jaw driver (108) and the jaw (110) as well as the means by which the cabling controls these elements. The jaw driver (108) has a cam feature (108f) which engages a cam surface (110a) of the jaw (110). The cam surface (110a) has front and back surfaces; the front surface drives the jaw to a proximately closed position, whereas the back surface is utilized when driving the jaw to a proximately open position. The cam surface (110a) is designed to produce a particular pattern of force amplification; at different positions of the jaw driver (108), a different mechanical advantage is obtained between the jaw driver (108) and jaw (110). The particular shape of the cam surface (110a) may be designed to produce different force amplification effects.

In general, producing one of the three motions of the end effector assembly (100) requires retracting two cables and relaxing two other cables. As shown in FIG. 13, opening the jaw (110) requires a counterclockwise rotation as viewed from the left of the jaw driver (108). This motion is produced by retracting cables X and Y and relaxing cables W and Z; this will be denoted in general as a XY/WZ motion. When cables X and Y are retracted, this produces no motion about the primary axes of the end effector assembly (100) because these cables are opposed with respect to both axes. Both of these cables act to rotate the jaw driver (108) in a counterclockwise direction, which in turn pulls cables W and Z to translate in a distal direction, producing the XY/WZ cable motion seen in FIGS. 13 and 14.

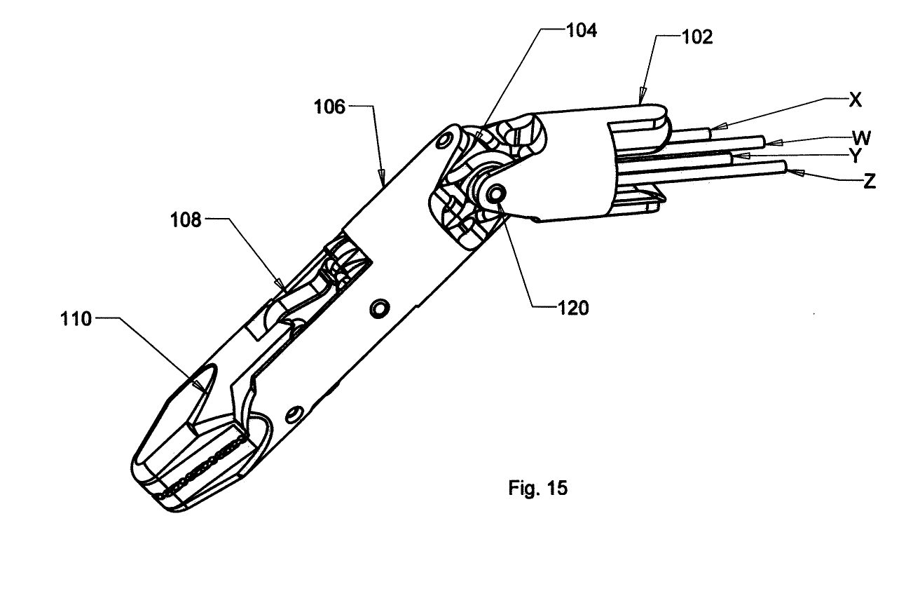

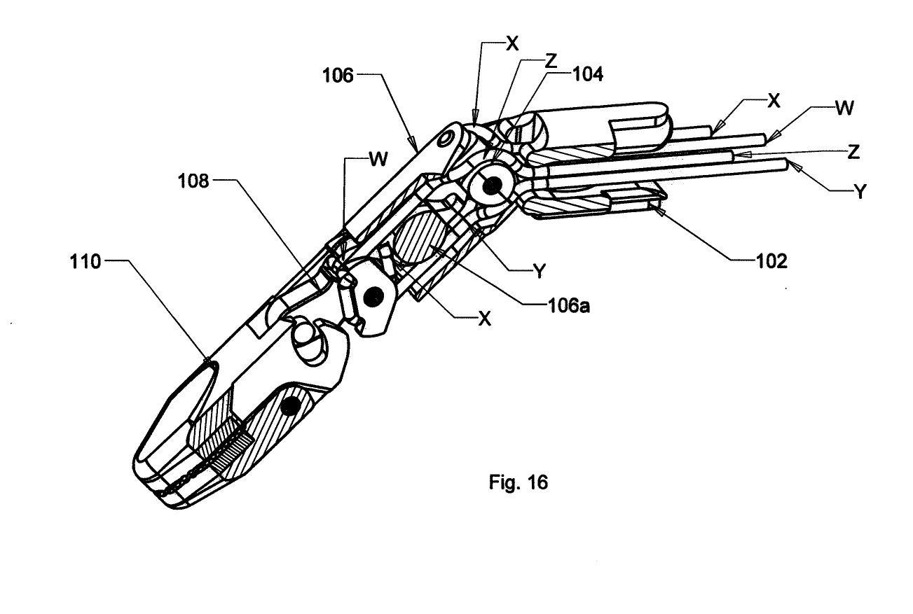

FIGS. 15 and 16 depict articulation of the end effector assembly (100) about its primary axis. This is produced by a WY/XZ motion. Cables W and Y are opposed with respect to the secondary axis of the end effector assembly (100) and the jaw driver (108) rotation. These cables thus produce motion about the primary axis of the end effector assembly (100) when retracted simultaneously. In response, cables X and Z are translated in a distal direction, producing the WY/XZ motion shown.

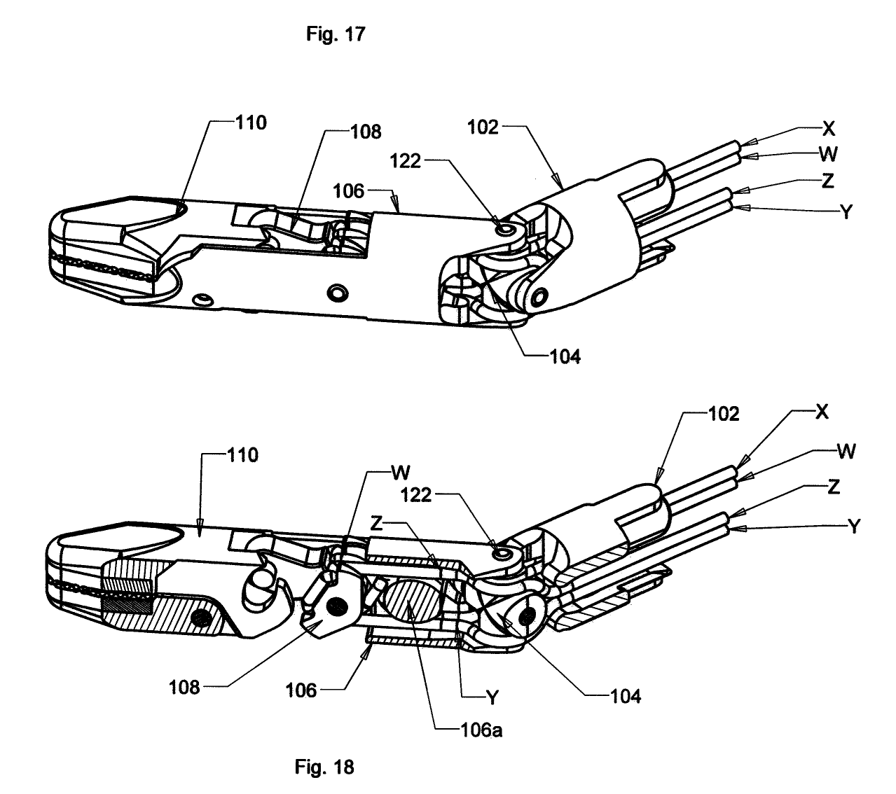

FIGS. 17 and 18 depict articulation of the end effector assembly (100) about its secondary axis. This is produced by a WX/YZ motion. Cables W and X are opposed with respect to the primary axis of the end effector assembly (100) and the jaw driver (108) rotation. These cables thus produce motion about the secondary axis of the end effector assembly (100) when retracted simultaneously. In response, cables Y and Z are translated in a distal direction, producing the WX/YZ motion shown. In each of the three previously described motions, the opposite action can be produced by opposite cable actuation. For example, the jaw is opened by a XY/WZ motion, and can thus be closed by a WZ/XY motion.

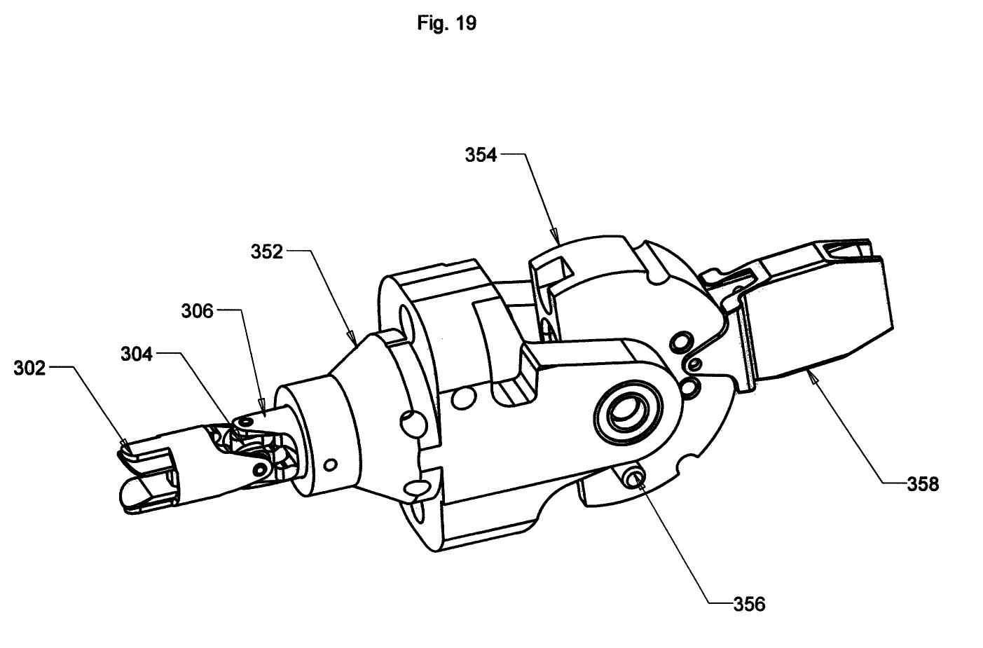

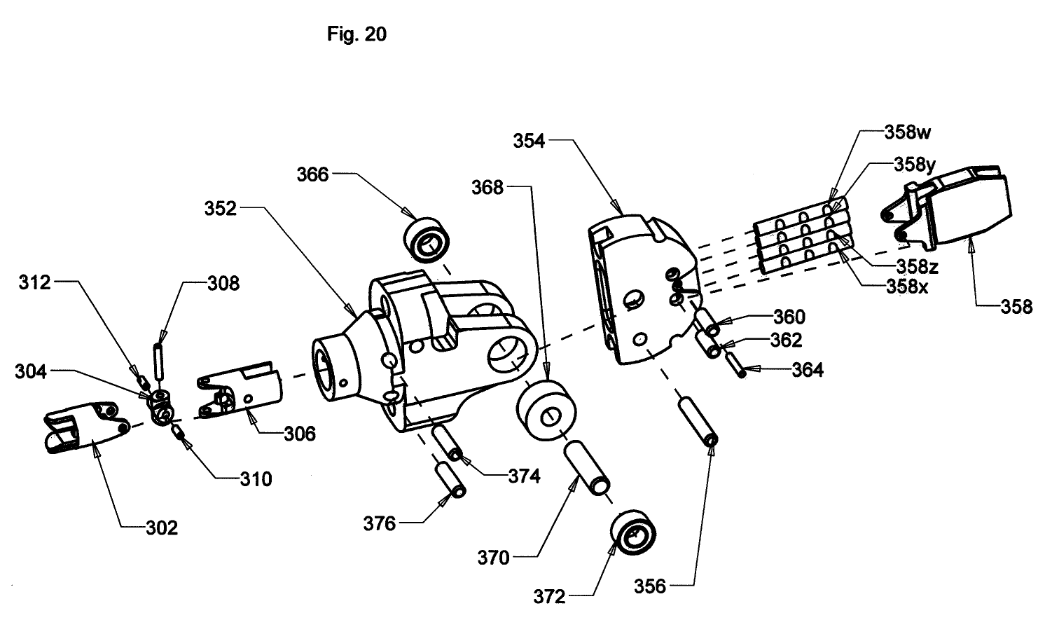

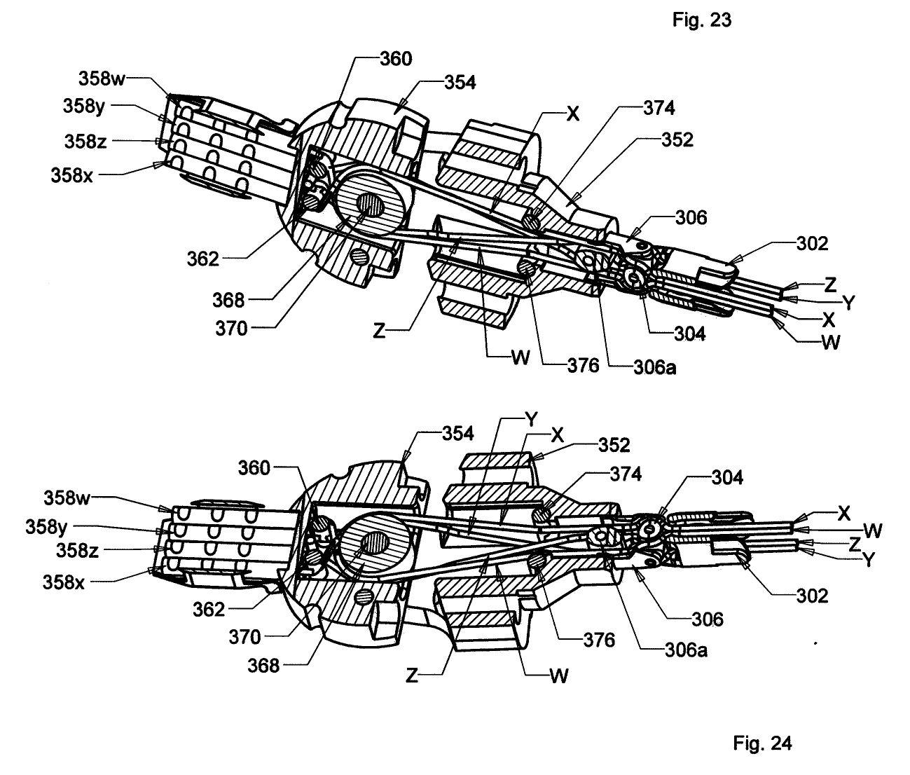

FIGS. 19-24 depict the structure and cabling of the proximal joint (300) and proximal control assembly (350). The proximal joint center element (304) is pivotally connected to the proximal joint base element (302) via two pins (310,312) that define a primary axis of articulation. The proximal joint end element (306) is pivotally connected to the proximal joint center element (304) via a pin (308) which defines a secondary axis of articulation. These pivotal connections may in general be made by any number of pins or pin features which define two perpendicular axes of articulation. The proximal joint end element (306) is connected to the proximal control base element (352). The tensioner element (354) is pivotally connected to the proximal control base element (352) via the tensioner pin (370) and tensioner bearings (366,372). The tensioner pulley (368) is also mounted on the tensioner pin (370). The tensioner element (354) also contains the tensioner drive pin (356), and cable guide pins (360,362). The cable crimp cover (358) is attached via a pin (364) to the tensioner (354) and houses the four cable crimps (358w,358x,358y,358z).

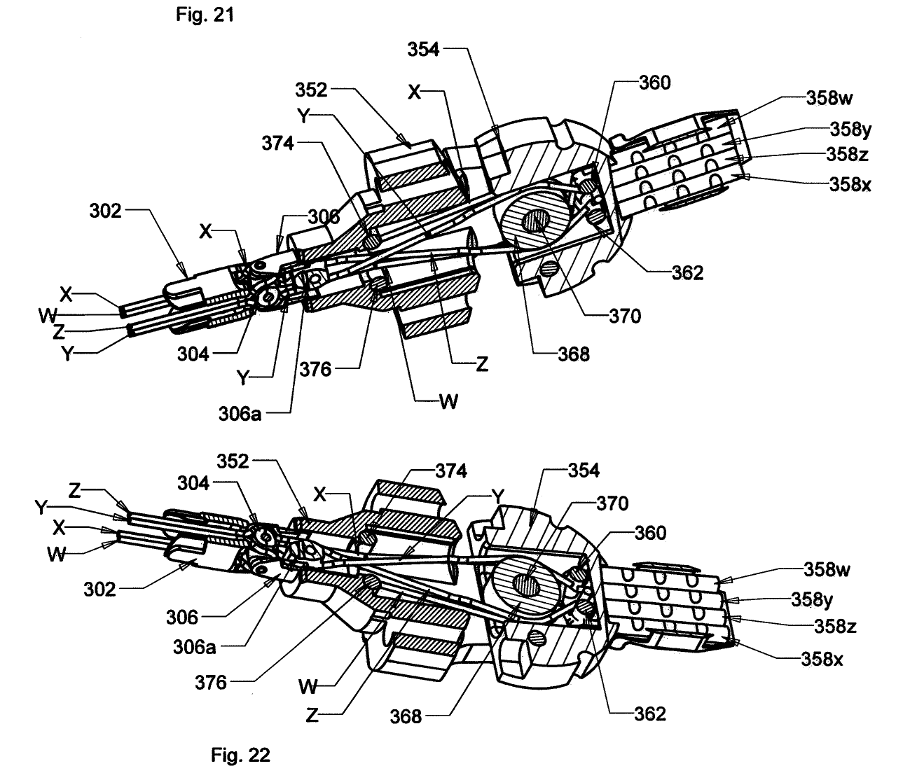

With specific reference to FIGS. 21-24, the details of the cabling within the proximal control assembly (350) are described herein. Cable W enters the proximal joint base (302) and passes underneath a horizontal round feature of the proximal center joint element (304) before passing around the right side of a vertical round feature of the proximal center joint element (304) and entering the proximal joint end (306). Cable X enters the proximal joint base (302) and passes over a horizontal round feature of the proximal center joint element (304) before passing around the right side of a vertical round feature of the proximal center joint element (304) and entering the proximal joint end (306). Cable Y enters the proximal joint base (302) and passes underneath a horizontal round feature of the proximal center joint element (304) before passing around the left side of a vertical round feature of the proximal center joint element (304) and entering the proximal joint end (306). Cable Z enters the proximal joint base (302) and passes over a horizontal round feature of the proximal center joint element (304) before passing around the left side of a vertical round feature of the proximal center joint element (304) and entering the proximal joint end (306). Cable W passes underneath the proximal joint end central guiding feature (306a), over a cable guide pin (376), under the tensioner pulley (368), over another cable guide pin (360), through the tensioner (354) and terminates in a crimp (358w). Cable X passes over the proximal joint end central guiding feature (306a), under a cable guide pin (374), over the tensioner pulley (368), under a cable guide pin (362), through the tensioner (354) and terminates in a crimp (358x). Cable Y passes underneath the proximal joint end central guiding feature (306a), over the tensioner pulley (368), under a cable guide pin (360), through the tensioner (354) and terminates in a crimp (358y). Cable Z passes over the proximal joint end central guiding feature (306a), under the tensioner pulley (368), over a cable guide pin (362), through the tensioner (354) and terminates in a crimp (358z).

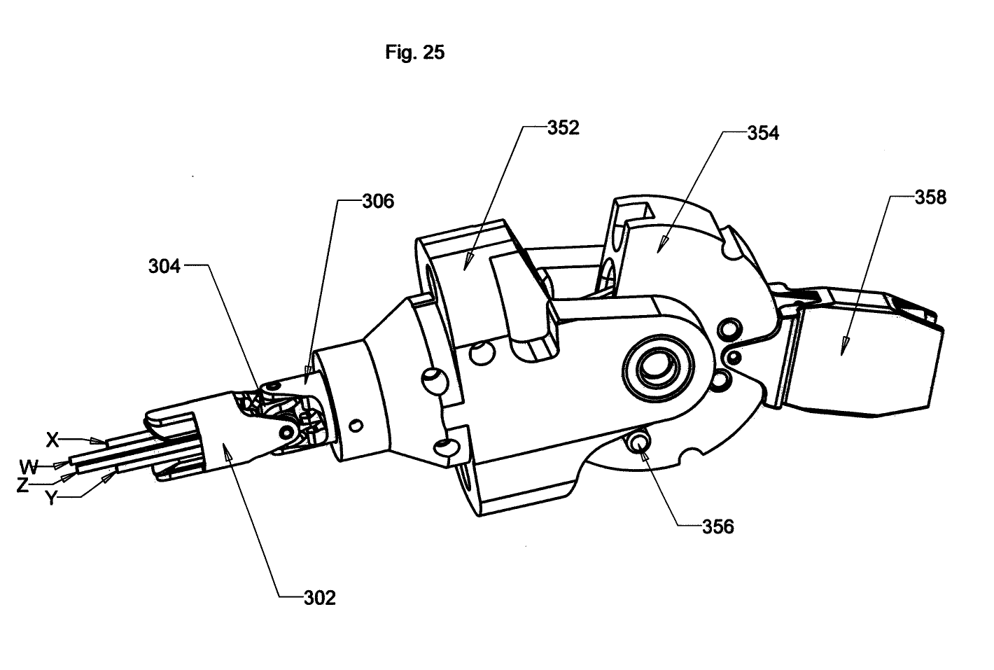

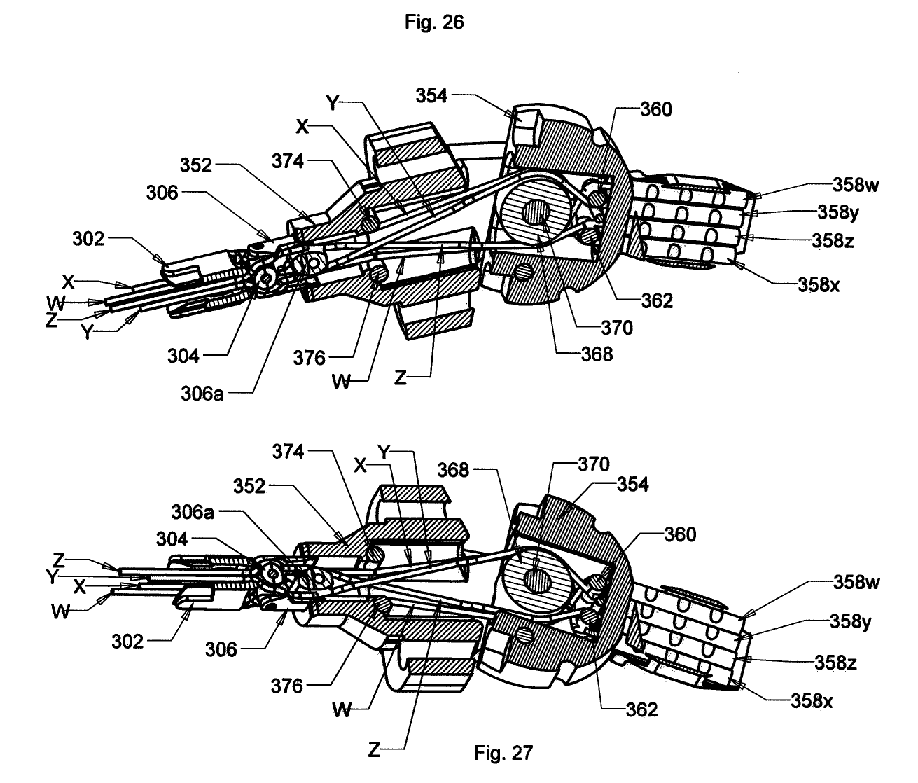

FIGS. 25-27 illustrate the means by which the proximal joint (300) and proximal control assembly (350) achieve the XY/WZ motion to open the jaw (110) as described previously. When the tensioner (354) is rotated in a clockwise direction as viewed from the left, the crimps (358x,358y) retain cables X and Y against the tensioner (354) and cause cables X and Y to be retracted as they are pulled around the tensioner pulley (368). Since these cables are opposed with respect to the first and second axes of articulation, they produce no effect on the proximal joint (300). Thus, cables X and Y are retracted, and cables W and Z are relaxed, producing the XY/WZ motion.

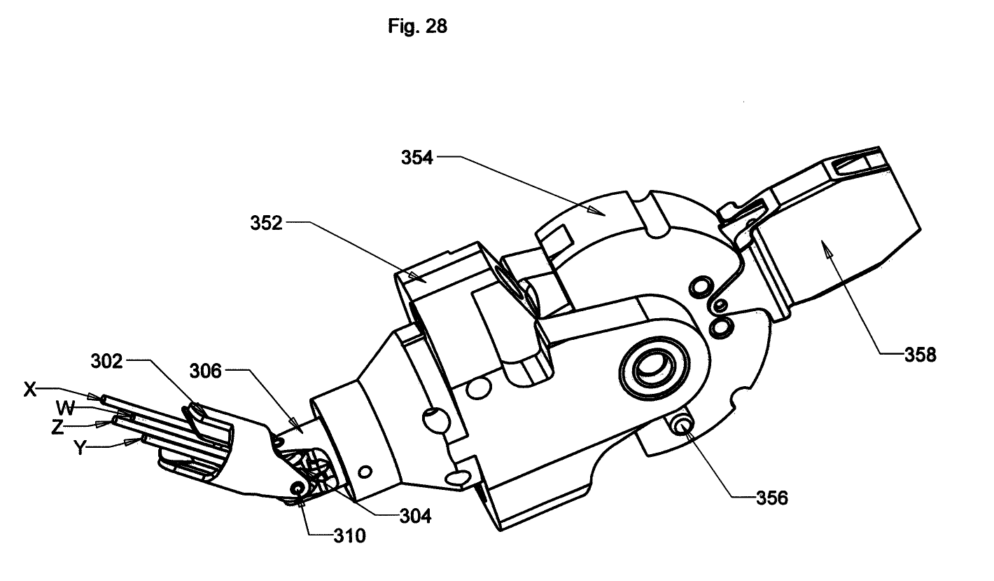

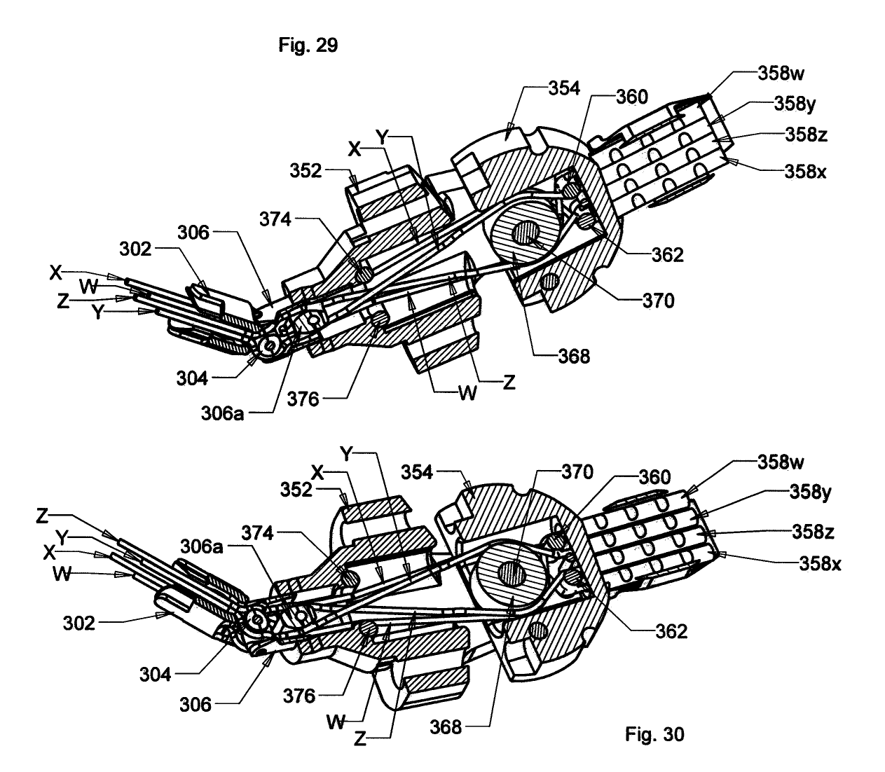

FIGS. 28-30 illustrate the means by which the proximal joint (300) and proximal control assembly (350) achieve the WY/XZ motion to articulate the end effector assembly (100) about a primary axis as described previously. When the proximal control assembly base (352) is rotated in a counterclockwise direction as viewed from the left about the proximal joint base's (302) primary axis of articulation, cables W and Y are retracted. Since these cables are opposed with respect to the secondary axis of articulation and the tensioner's (354) axis of rotation, this has no effect on those elements. The corresponding relaxation of cables X and Z acts with this retraction to produce the WY/XZ motion.

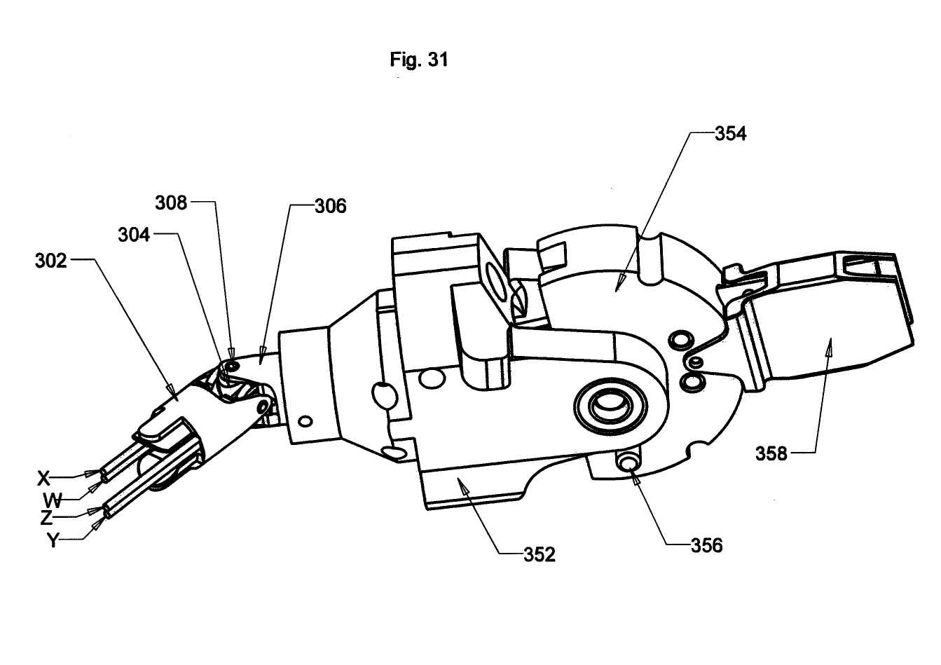

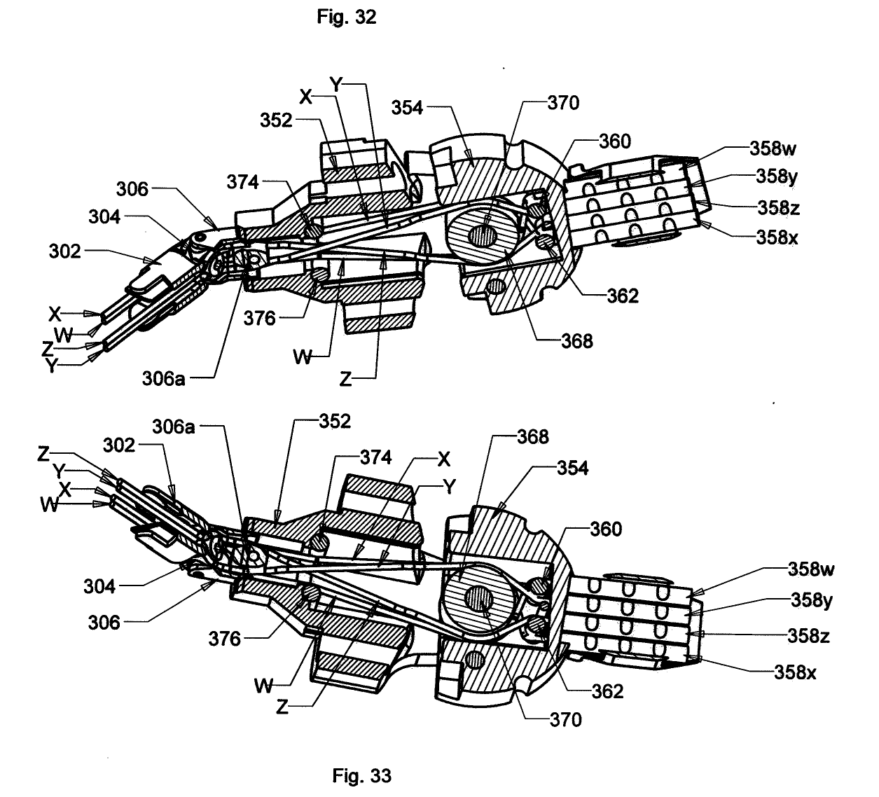

FIGS. 31-33 illustrate the means by which the proximal joint (300) and proximal control assembly (350) achieve the WX/YZ motion to articulate the end effector assembly (100) about a secondary axis as described previously. When the proximal control assembly base (352) is rotated in a clockwise direction as viewed from above about the secondary axis of articulation, cables W and X are retracted through the proximal joint (300). Since these cables are opposed with respect to the primary axis of articulation and the tensioner's (354) axis of rotation, this has no effect on those elements. The corresponding relaxation of cables Y and Z acts with this retraction to produce the WX/YZ motion.

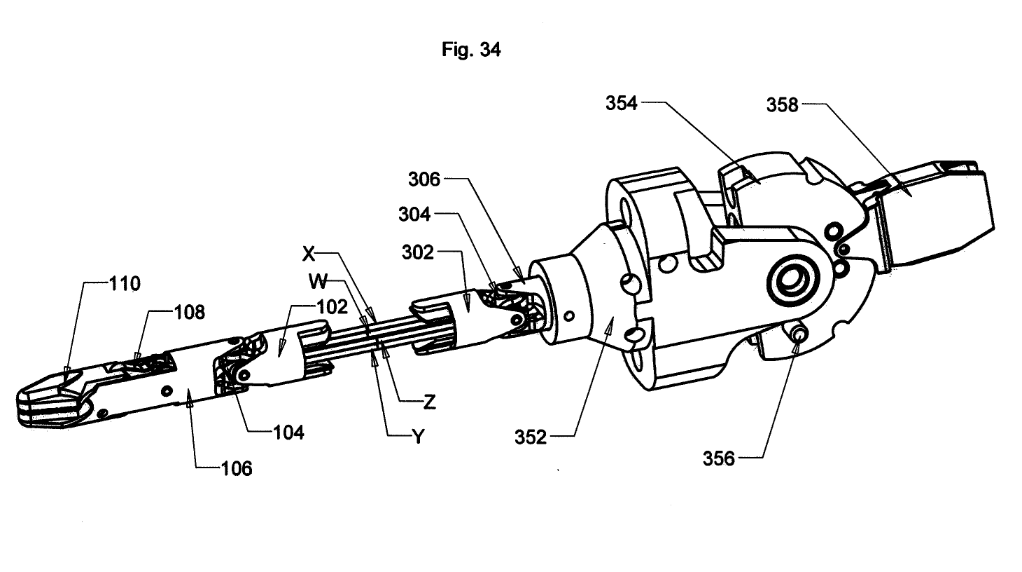

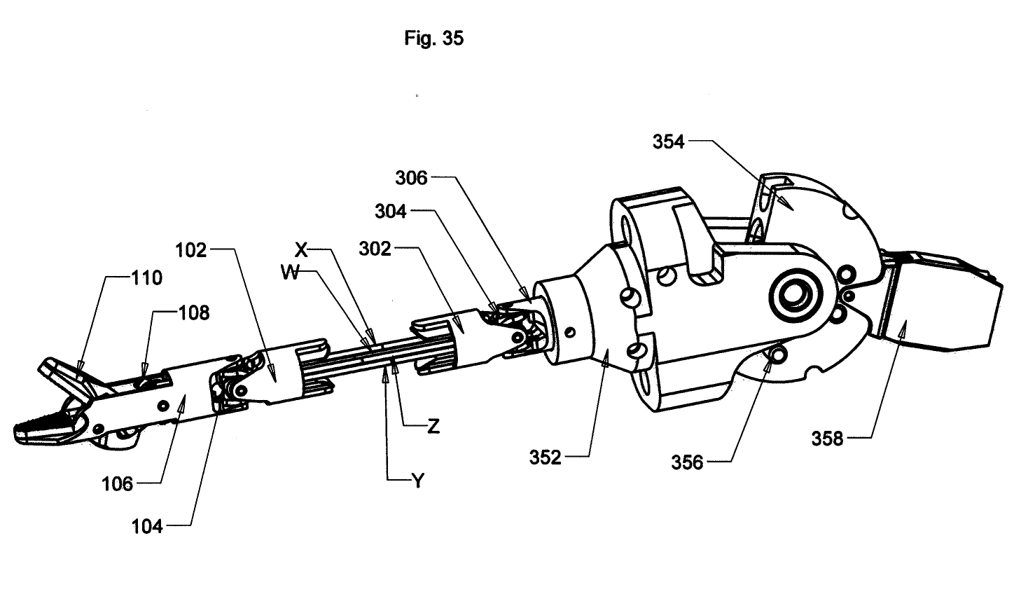

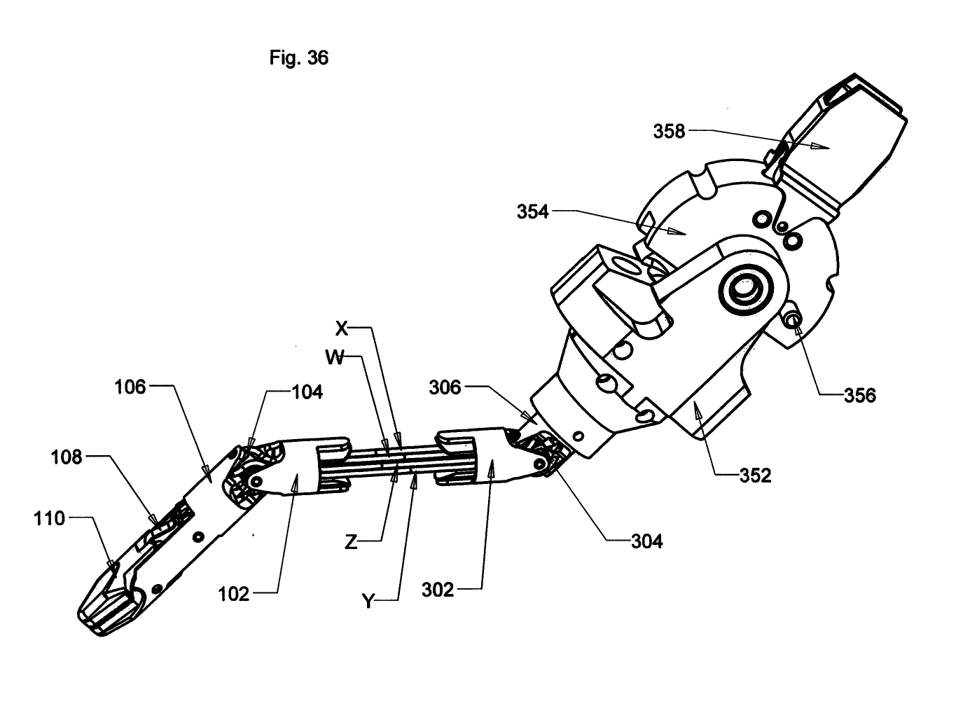

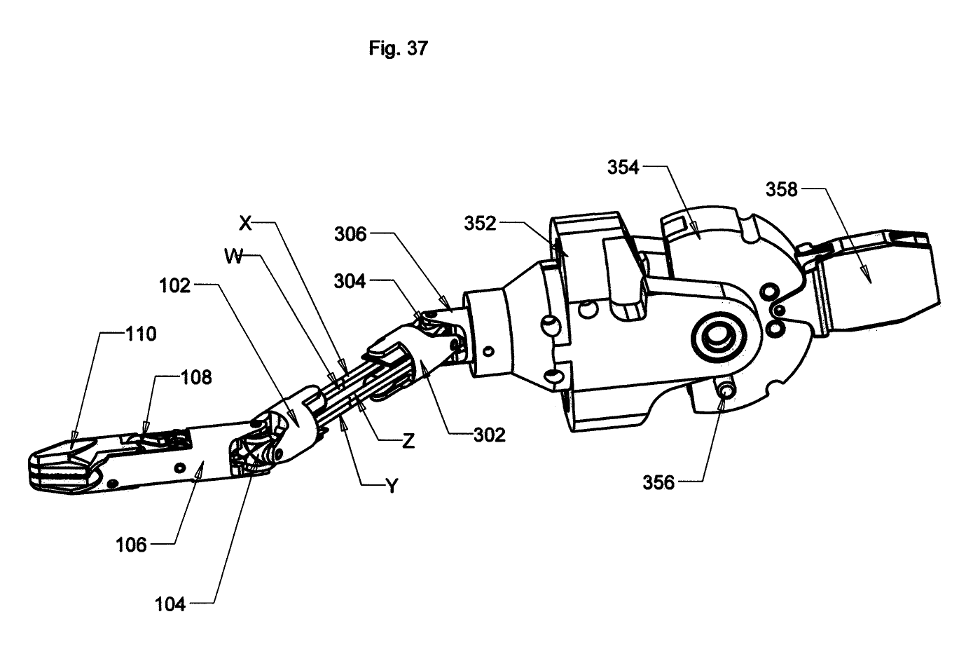

FIGS. 34-37 depict the aggregate effect of the motions described previously that produce the three primary motions of the end effector assembly (100) as controlled by the proximal joint (300) and proximal control assembly (350). In the presently described embodiment of the subject invention, cables W, X, Y, and Z all pass through the tube (202) and detensioner assembly (210) between the end effector assembly (100) and proximal joint (300) and proximal control assembly (350). In these figures, the tube (202) and detensioner assembly (210) are not show; this shows a simplified schematic representation of the cabling within the device. In each case, two cables are retracted and two are relaxed; the distal translation of cables is matched by the cable retraction such that there is no net gain or loss of tension in these cables in the current embodiment. FIG. 35 specifically depicts the rotation of the tensioner (354) which causes a XY/WZ motion which in turn rotates the jaw driver (108) and subsequently the jaw (110) into a proximately open position. FIG. 36 depicts the articulation of the proximal joint (300) and proximal control assembly (350) about its primary axis, thus producing a WY/XZ motion, which in turn causes the end effector assembly (100) to articulate about its primary axis. FIG. 37 depicts the articulation of the proximal joint (300) and proximal control assembly (350) about its secondary axis, thus producing a WX/YZ motion, which in turn causes the end effector assembly (100) to articulate about its secondary axis. All combinations of these three motions may in turn be produced by combinations of the controlling movements of the proximal joint (300) and proximal control assembly (350).

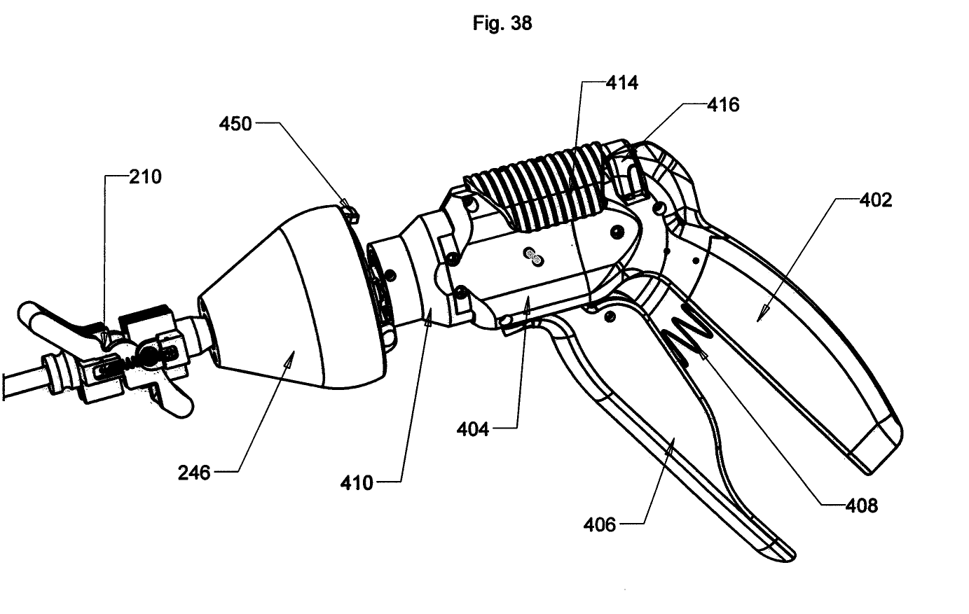

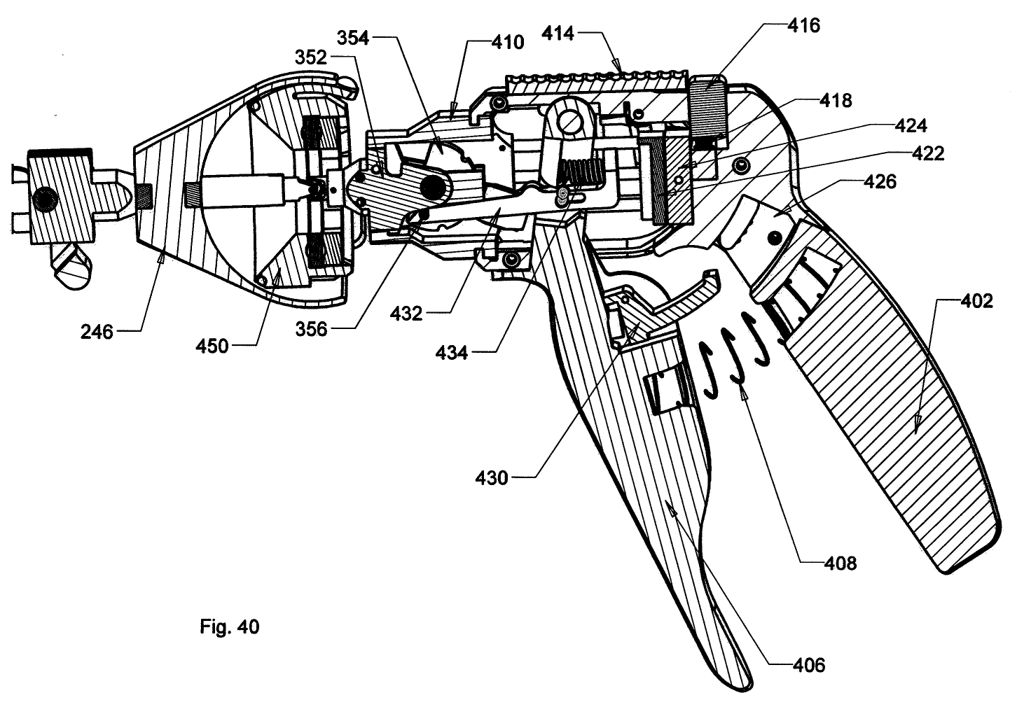

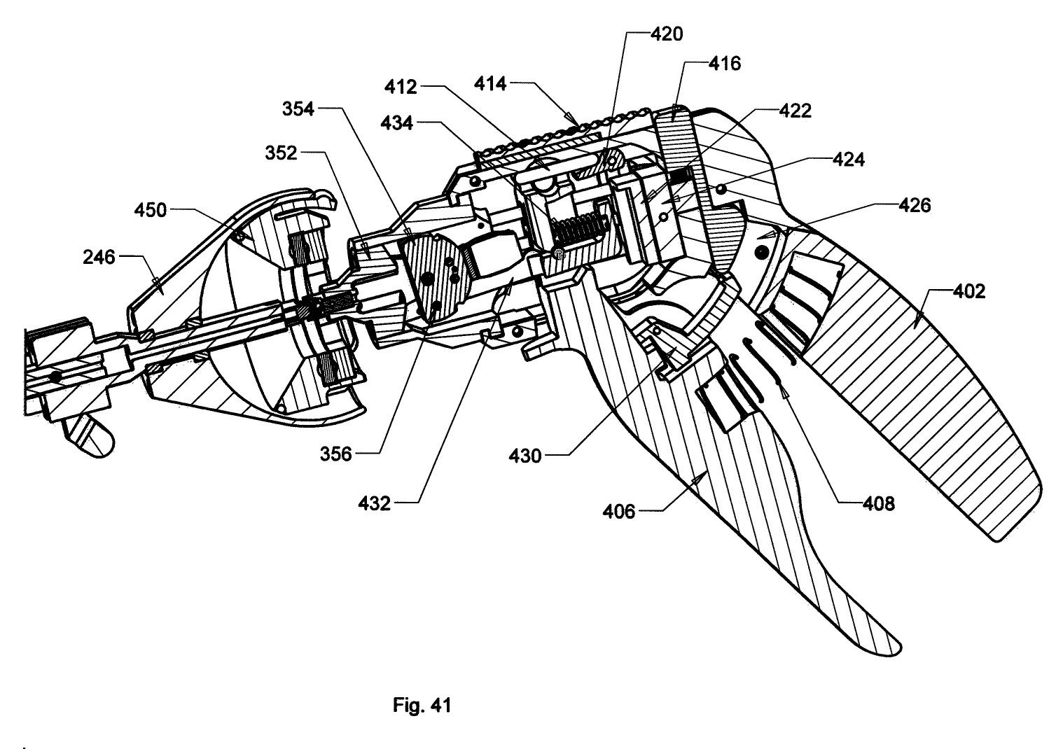

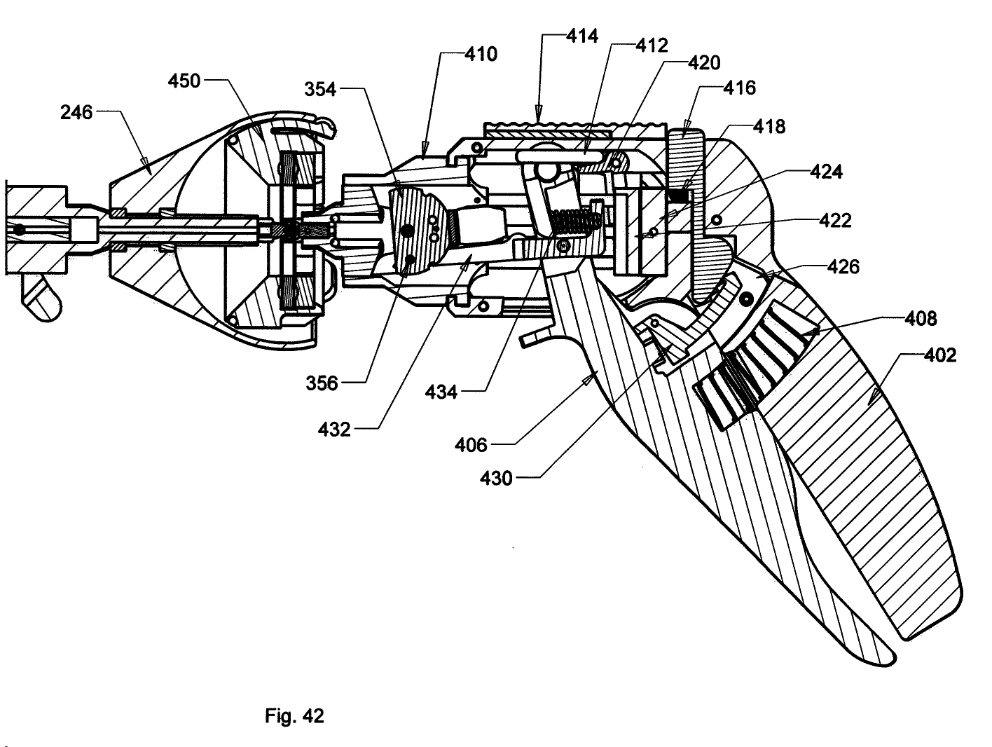

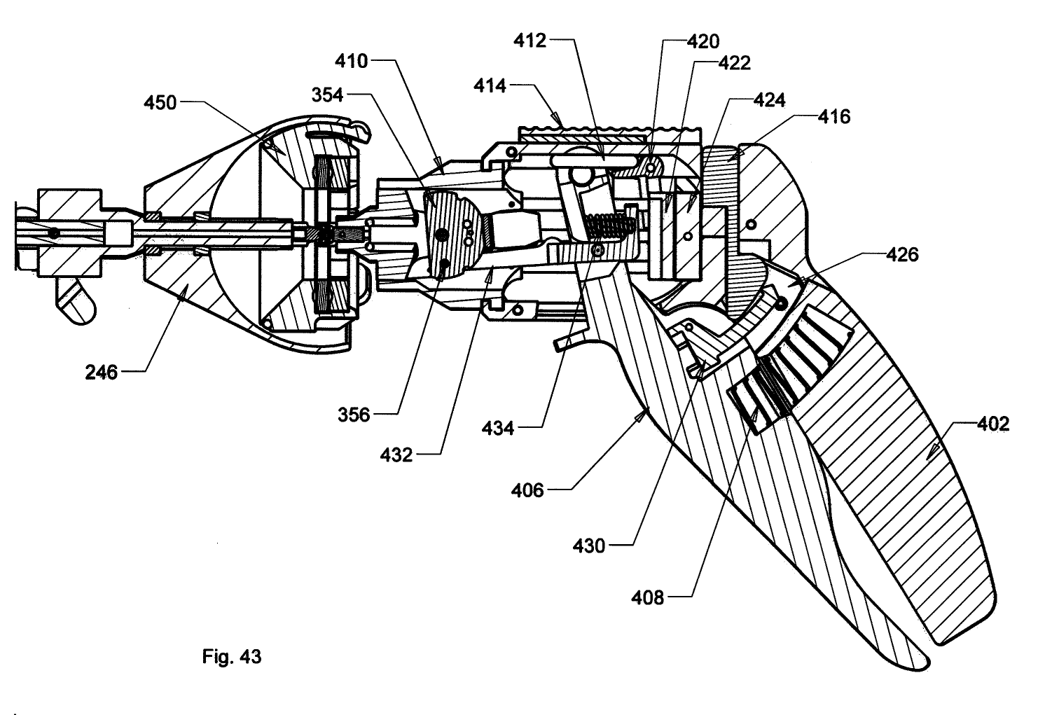

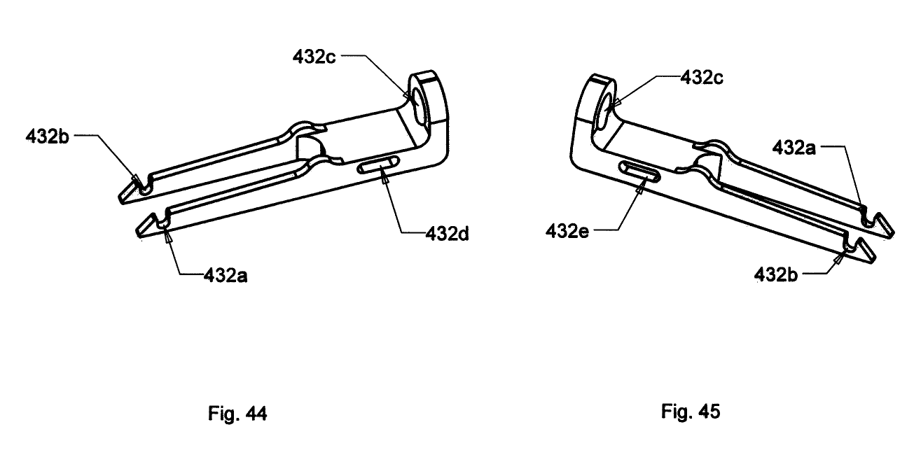

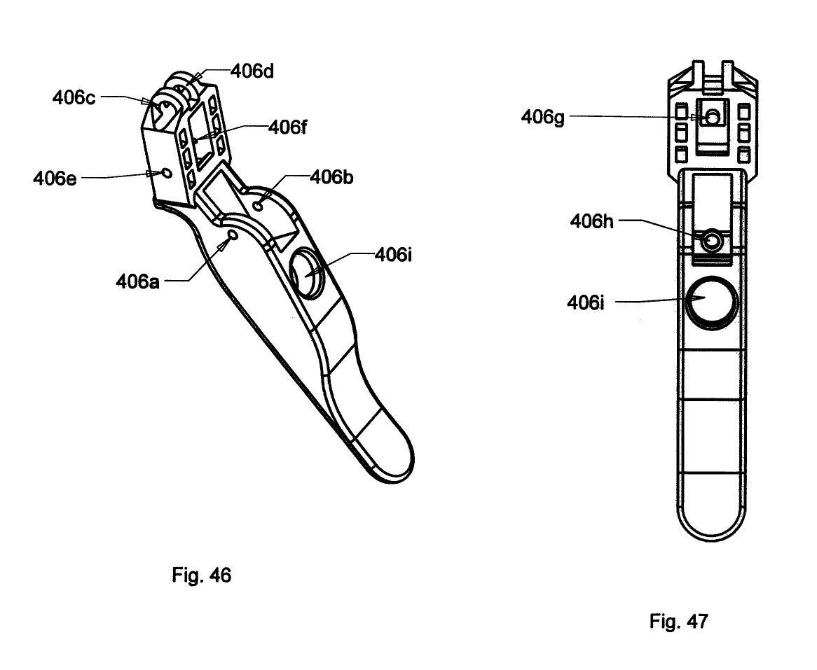

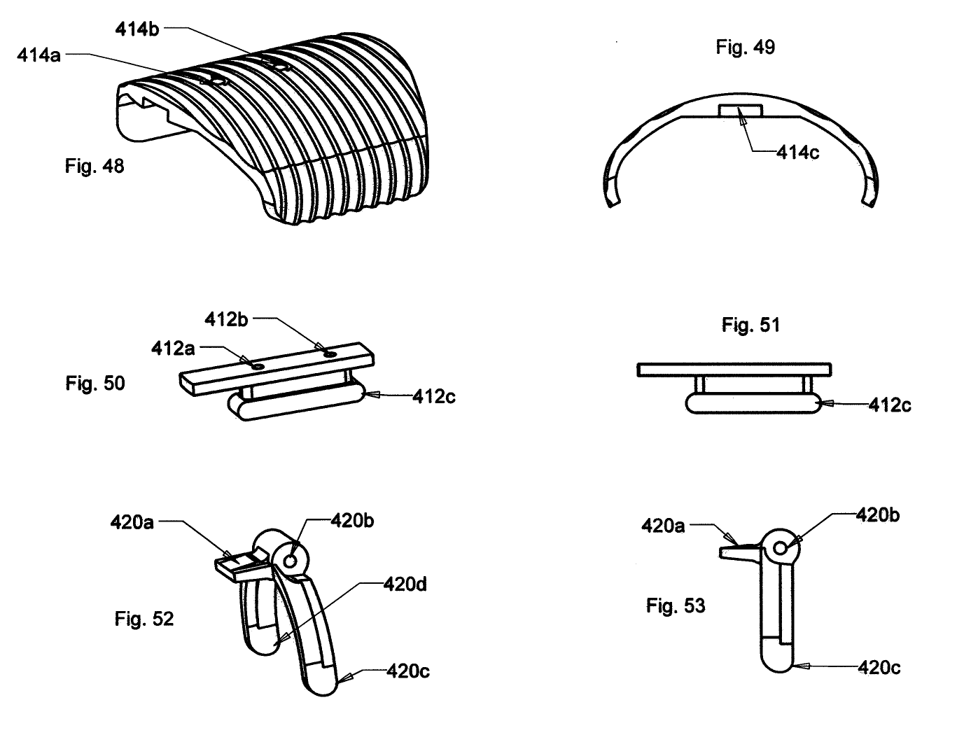

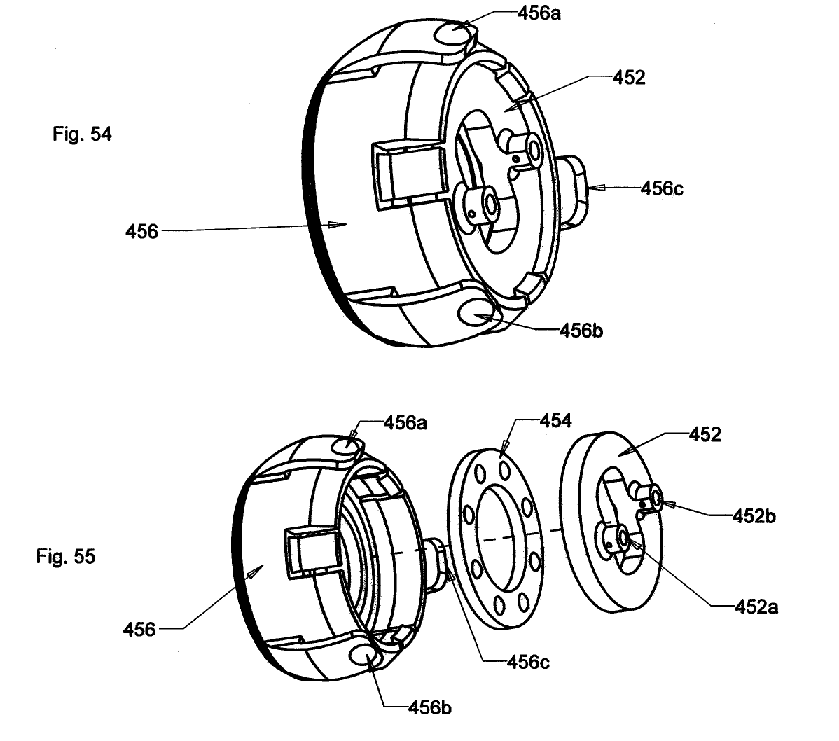

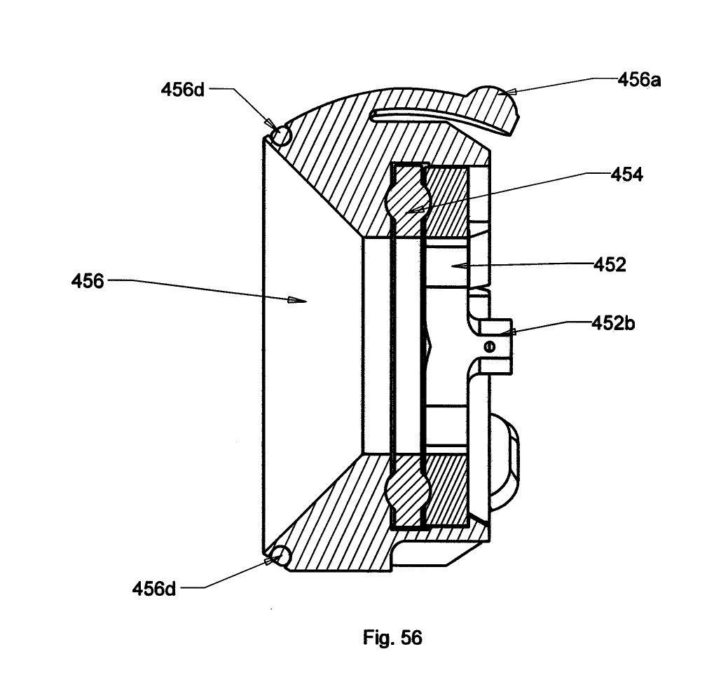

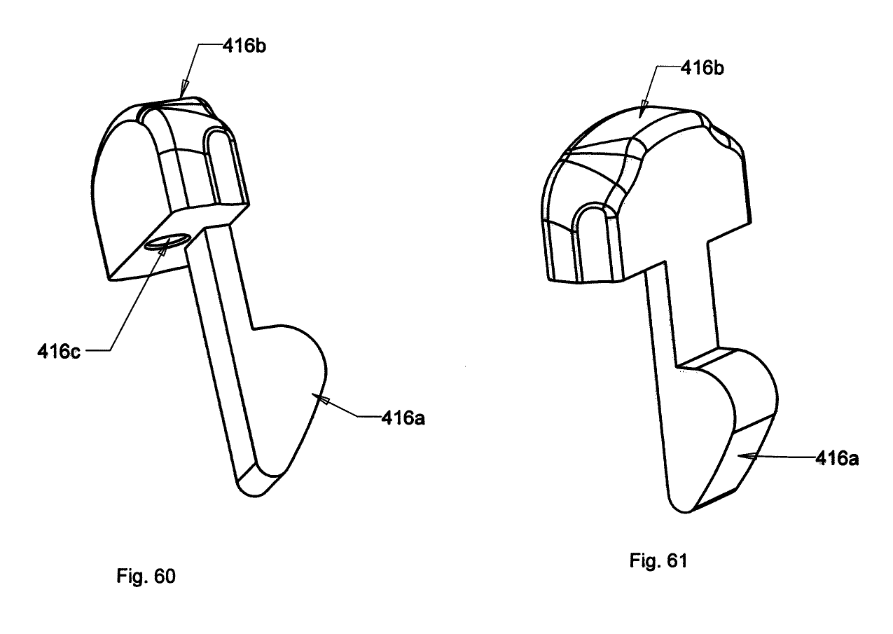

FIGS. 38-61 depict the handle (400) and its components. The handle (400) contains a base element (402) and cover element (404) with trigger element (406) pivotally mounted within via two features (406c,406d). The trigger (406) is biased by a spring (408) mounted within a pocket feature (406i) to a proximately open position. With specific reference to FIGS. 40-42, the means by which the cartridge (200) interfaces with the handle (400) is described herein. The proximal control base element (352) is detachably connected to the handle adapter element (410). This connection may be achieved by a variety of means, including but not limited to a friction fit, latch mechanism, or removable screws or pins. The tensioner drive pin (356) is actuated by the trigger latch element (432). FIGS. 44-47 detail the connection features of the trigger (406) and trigger latch (432). The trigger latch element (432) is connected to the trigger (406) via two screws (433,435) which are mounted in holes (406e,406f) and subsequently enter two slots (432d,432e) in the trigger latch element (432). This connection allows the trigger latch (432) to rotate as well as translate in a front/back direction with respect to the trigger (406). A trigger clutch spring (434) biases the trigger latch (432) to a rear position and applies a torque that locks the trigger latch (432) against the tensioner drive pin (356). This clutch spring (434) is mounted around a round feature (406g) of the trigger (406) and within a pocket feature (432c) of the trigger latch (432). The trigger latch (432) engages the tensioner drive pin (356) via two slot features (432a,432b). The slot features (432a,432b) provide a removable pivotal connection between the trigger latch (432) and the tensioner drive pin (356). Linear motion of the trigger latch (432) is translated into rotational motion of the tensioner (354) via the tensioner drive pin (356). FIG. 40 shows the trigger (406) in a proximately open position; this corresponds to an angular position of the tensioner (354) that causes the jaw (110) to be in a proximately open position. FIG. 41 shows the trigger (406) in a position that corresponds to an angular position of the tensioner (354) that causes the jaw (110) to be in a proximately closed position. When the trigger (406) is closed beyond the position that has closed the jaw (110), as seen in FIG. 42, the trigger clutch spring (434) compresses, applying additional force to close the jaw (110). The trigger clutch spring (434) acts to regulate the amount of force that may be applied to the jaw (110).

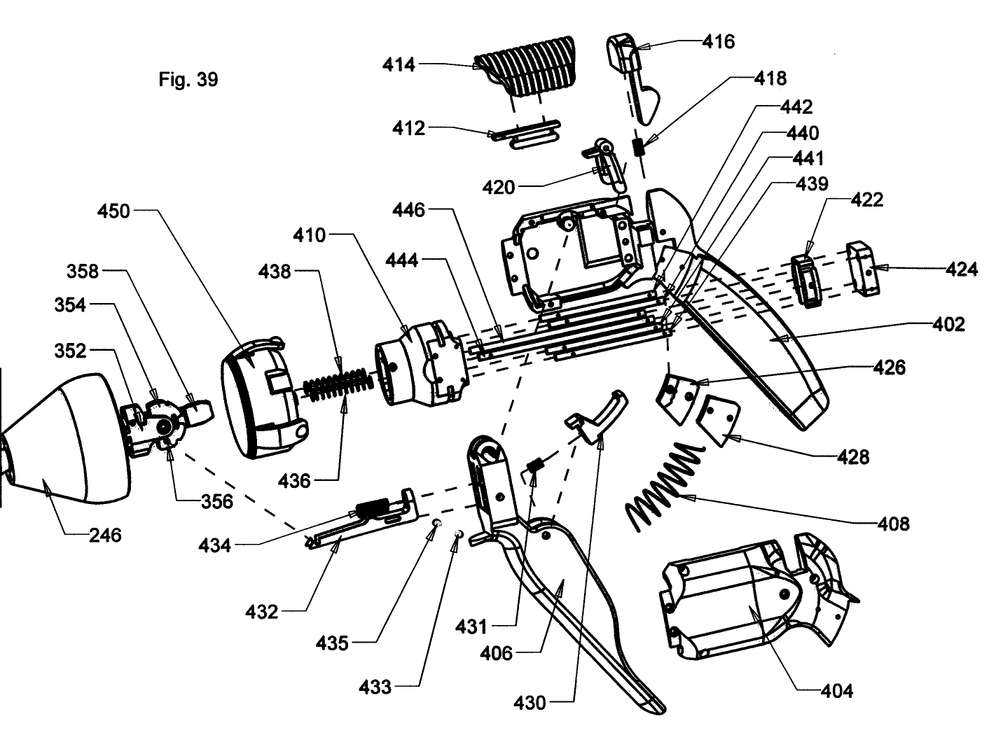

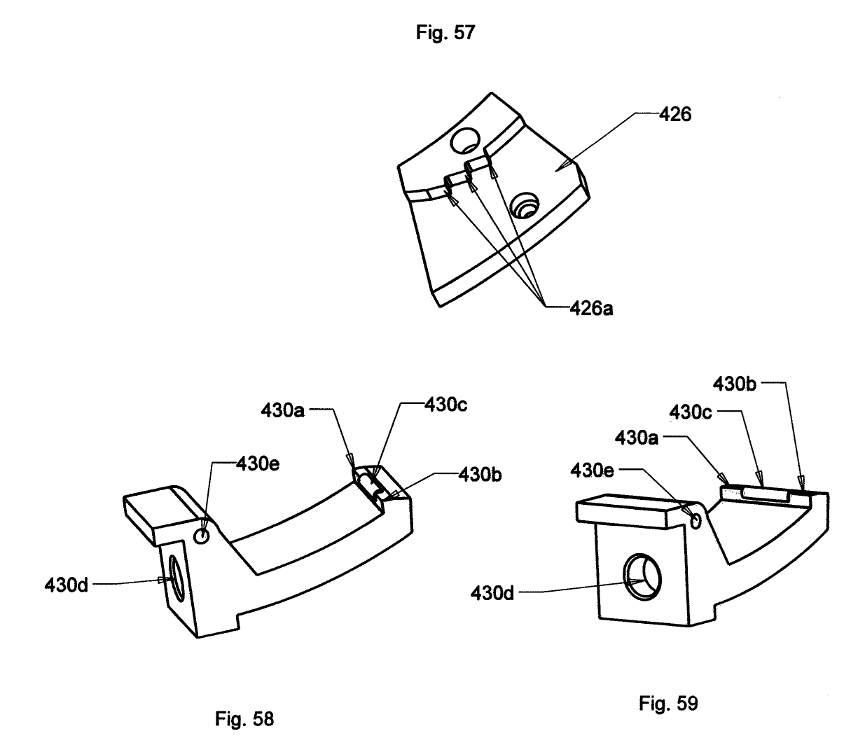

With specific reference to FIGS. 39, 42, 43, 46, 47 and 57-61, the operation of the trigger (406) is further described herein. A ratchet latch (430) is pivotally mounted to the trigger (406) at connection points (406a,406b, 430e) on the trigger (406) and ratchet latch (430). The ratchet latch (430) is biased into a locking position by a spring (431) mounted within pocket features (430d,406h) on the ratchet latch (430) and trigger (406). When the trigger (406) is closed, tooth features (430a,430b) on the ratchet latch (430) engage two ratchet plates (426,428). The second ratchet plate (428) is a mirror image of the first ratchet plate (426); thus, only the details of the first ratchet plate (426) are shown. FIG. 57 details the first ratchet plate (426) and shows the tooth features (426a) that engage the ratchet latch (430). The ratchet release element (416) is biased into a disengaged position by a spring (418) mounted in a pocket feature (416c). When the ratchet release element (416) is depressed by engaging its top surface (416b), this spring (418) compresses, and the cam surface (416a) of the ratchet release (416) engages the cam surface (430c) of the ratchet latch (430), causing the ratchet latch (430) to disengage the ratchet plates (426,428). This releases the trigger (406).

With specific reference to FIGS. 39, 42, and 48-56, the function of the brake assembly (450) and associated braking function of the handle (400) is described herein. The brake assembly (450) is biased to an engaged position by two springs (436,438) on two shafts (444,446). These shafts (444,446) control the linear movement of the brake assembly (450) and are in turn controlled by the brake slide element (422). The brake slide element (422) is constrained to move linearly in a forward/backward direction by four guide rails (439,440,441,442) that are mounted in the handle adapter (410) and guide rail mount (424). The brake actuation element (420) is pivotally mounted in the handle base (402) and handle cover (404) via a hole (420b) which may be used for a pin, screw, pin feature, or other pivotal connection means. Cam features (420c,420d) engage a flange on the brake slide element (422) and allow rotation of the brake actuation element (420) to cause a translational movement of the brake slide element (422) toward the proximal end of the instrument. This rotation is caused by a cam engagement between a round feature (412c) of the brake control element (412) and a cam surface (420a) of the brake actuation element (420). The brake control element (412) is contained with in a slot (414c) of the brake grip element (414) and attaches at two connection points (412a,412b,414a,414b). The brake grip element (414) allows the user to control the translational movement of the brake control element (412) which in turn controls the rotation of the brake actuation element (420) and subsequently the translational movement of the brake slide element (424).

The brake assembly (450) is translated linearly in a forward/backward direction, and in doing so engages and disengages the proximal joint block (246) which is mounted on bearings (242,244). The bearings (242,244) allow the distal portion of then instrument to rotate freely of the proximal joint block (246). The joint brake assembly (450) is composed of a joint brake element (456), thrust bearing (454), and joint brake collar (452). The joint brake collar (452) has two pocket features (452a,452b) at which the joint brake assembly (450) connects to the two shafts (444,446) which control its linear movement. The joint brake element (456) has three detent features (456a,456b,456c) which engage the edge of the proximal joint block (246) when the handle (400) is in a proximately centered position. This provides a soft lock at a proximately centered position. These detent features (456a,456b,456c) intermittently engage the interior surface of the proximal joint block (246) providing resistance to motion of the handle (400) which stabilizes the instrument. The joint brake element (456) also contains a gasket feature (456d) which locks the articulation of the handle (400) when the joint brake assembly (450) is pressed against the proximal joint block (246) by the joint brake springs (436,438). Even when articulation is locked, the thrust bearing (454) and joint block bearings (242,244) permit axial rotation of the handle (400) which translates to axial rotation of the jaw base (106) and all mechanisms contained therein.

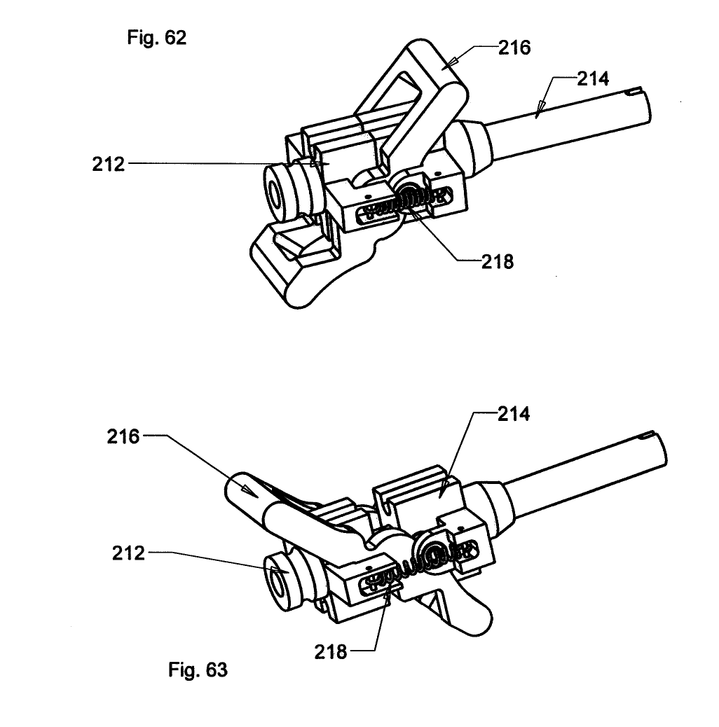

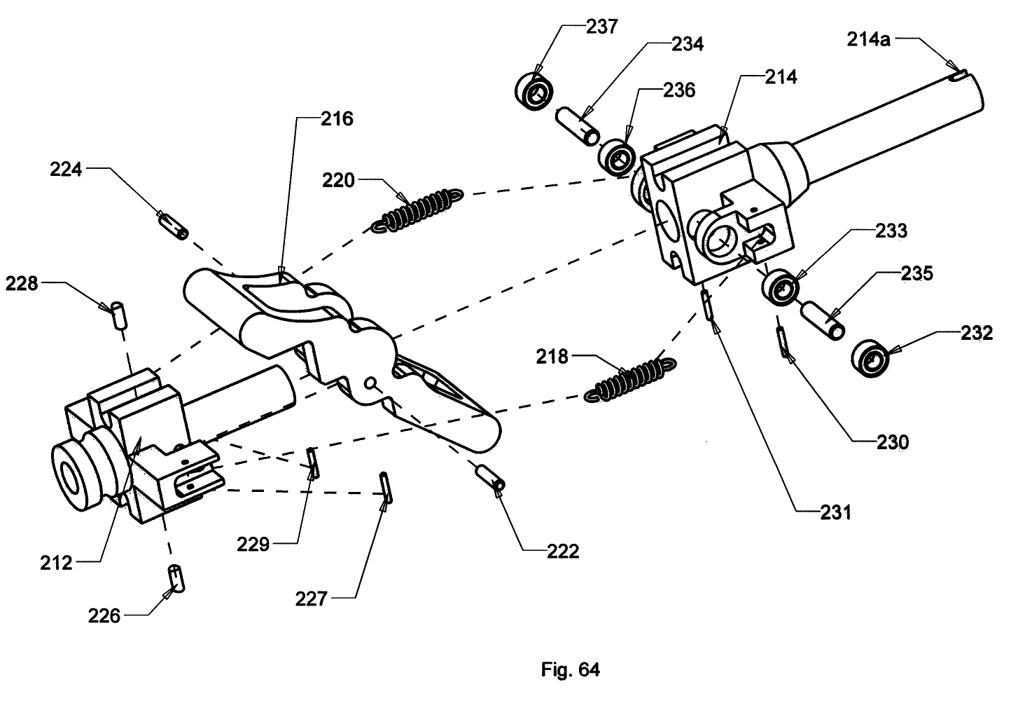

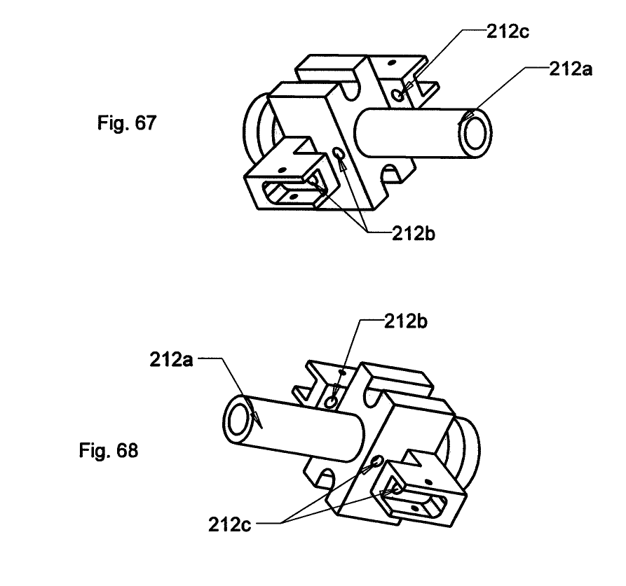

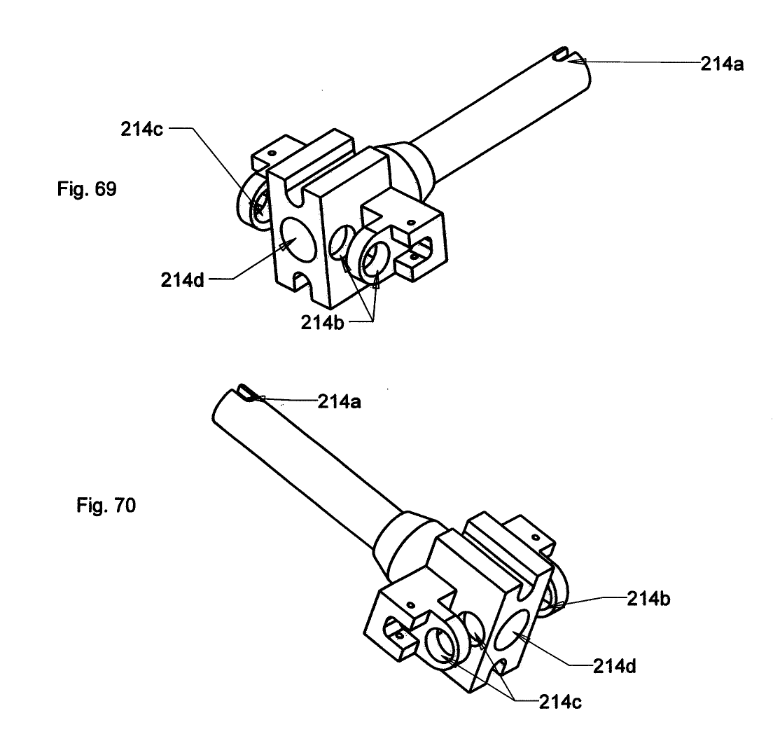

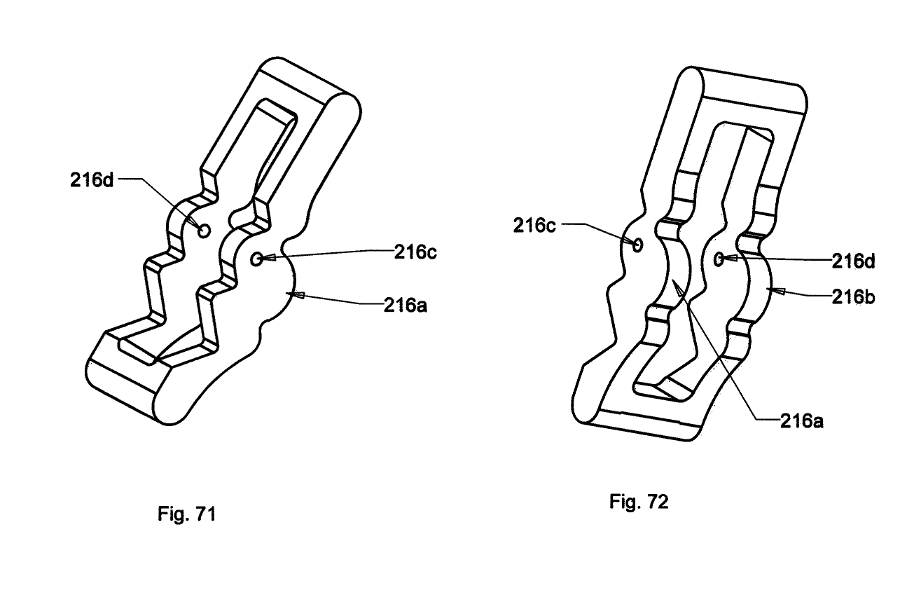

FIGS. 62-72 detail the components and function of the detensioner assembly (210). The detensioner assembly (210) contains a detensioner end element (214) mounted on a detensioner base element (212). The detensioner base element (212) engages the detensioner end element (214) via a protrusion (212a) that engages an interior surface (214d) and allows for linear movement of the detensioner end element (214) relative to the detensioner base element (212). This movement allows the detensioner assembly (210) to shift between proximately expanded and proximately compressed positions. A detensioner arm (216) engages the detensioner base (212) in a pivotal connection via two pins (222,224) which engage hole features (216c,216d) in the detensioner arm (216) and hole features (212b,212c) on the detensioner base (212). Two springs (218,220) bias the detensioner assembly (210) into a proximately compressed position, and are anchored at their ends by pins (227,229,230,231). Expansion of the detensioner assembly (210) is achieved by the interaction of the detensioner arm (216) and two cam pins (234,235) mounted on bearings (232,233,237,236) within hole and pocket features (214b,214c) in the detensioner end (214). Two pins (226,228) engage the end (202a) of the tube (202). A cutout feature (214a) at the end of the detensioner end (214) engages the proximal joint base (302).

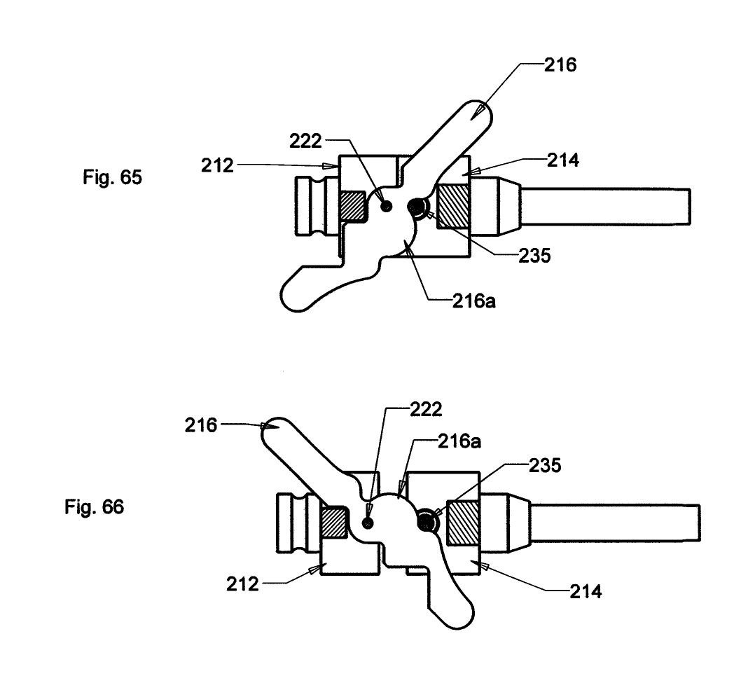

The engagement of the cam surfaces (216a,216b) of the detensioner arm (216) is detailed in FIGS. 65-66. When the detensioner arm (216) is rotated in a proximately counterclockwise direction as viewed from the left, the cam surfaces (216a,216b) drive the detensioner end (214) to the right via their engagement with the cam pins (234,235). This applies an equal marginal amount of tension to all control cables. During assembly, the detensioner (210) would likely be in an expanded configuration. After pre-loading was applied to all control cables, and the cables were secured, the detensioner (210) would be switched to a compressed configuration, relieving the tension on the control cables. This would extend the shelf life of the cables.

In the previously described figures, the end effector assembly (100) articulated in the opposite direction of the handle assembly (400) and proximal joint (300). This maintains a constant orientation of the end effector assembly (100) relative to the handle assembly (400), providing simple control to the user. The degree of articulation shown in these figures is meant for demonstrative purposes and is not an indication of any limitation of the design. The design of the end effector assembly (100) in this embodiment is meant to be generalized to any assembly utilizing four cables for actuation which achieves two degrees of articulation about perpendicular coplanar axes and a third degree of motion defined by another element designed to interact with the surgical environment; possible elements include but are not limited to cauterizing contacts, pliers, and scissor blades.

While the materials of the instrument are not intended to be constrained by this description, in application it is likely that the majority of the parts would be made from stainless steel or plastic. The end effector assembly (100), proximal joint (300) and tube (200) would be made from steel. The handle assembly (400) would be composed of hard plastic and metal components. The control cables would either be stainless steel rope, aramid fiber cables, or aligned polymer fiber cables.

We claim:

1. A surgical tool for use by an operator, comprising:
a manipulator adapted to receive at least a portion of a hand of the operator;
a proximal universal joint having a first end and a second end, the first end of the proximal universal joint being mounted to the manipulator;
a hollow elongated member having a first end, a second end, and a longitudinal axis, the first end of the elongated member being mounted to the second end of the proximal universal joint;
a distal universal joint having a first end and a second end, the first end of the distal universal joint being mounted to the second end of the elongated member; and
an end effector comprising
a universal joint element pivotally mounted to the second end of the distal universal joint for rotation about a first axis, and
a base member pivotally connected to the joint element for rotation about a second axis perpendicular to the first axis,
wherein pivoting of the first end of the proximal universal joint causes the end effector to move in a corresponding motion.

2. The surgical tool of claim 1, further comprising cabling operatively coupling the proximal joint and the end effector.

3. The surgical tool of claim 2, wherein the cabling comprises four cables that each engage the end effector.

4. The surgical tool of claim 1, wherein the end effector further comprises a digit pivotally mounted to the base member for movement relative to the base member between a closed position contacting the base member and an open position spaced from the base member.

5. The surgical tool of claim 4, wherein the end effector further comprises a driver pivotally mounted to the base member for movement relative to the base member, the driver including a cam element, wherein the digit defines an elongated opening for receiving the cam element such that the digit is movable between the closed position and the open position by movement of the driver relative to the base member.

6. The surgical tool of claim 5, wherein the opening in the digit is arcuate for varying the force at different relative positions of the digit and the driver.

7. The surgical tool of claim 5, further comprising cabling operatively coupling the proximal joint and the driver.

8. The surgical tool of claim 7, wherein the cabling comprises four cables that each engage the end effector.

9. The surgical tool of claim 1, wherein the manipulator comprises an actuator operable to control the end effector.

10. The surgical tool of claim 9, wherein the actuator comprises a trigger assembly adapted to be operable with a finger of the operator, wherein actuating the trigger assembly causes the driver to move relative to the base member.

11. The surgical tool of claim 1, wherein the proximal and end effector universal joints each comprise a proximal end member and distal end member, with each end member including a base portion and opposing arms extending from the base portion, the proximal end member and the distal end member mounted to a center block for each joint, the center block pivotable around two substantially coplanar, perpendicular axes, wherein the base portions and center block define openings for receiving the end effector control cables.

12. The surgical tool of claim 1, wherein the proximal and end effector universal joints are controlled by universal joint control cables anchored in the manipulator and may which be adjusted with means for tensioning the universal joint control cables.

13. The surgical tool of claim 1, wherein the manipulator has a longitudinal axis and a first angular position, and the end effector has a longitudinal axis parallel to the longitudinal axis of the manipulator and a first angular position, and
wherein at all relative positions of the manipulator and the end effector, the longitudinal axis of the manipulator and the longitudinal axis of the end effector remain parallel, and the degree of rotation of the manipulator about the longitudinal axis of the manipulator from the first angular position of the manipulator is equal to the degree of rotation of the end effector about the longitudinal axis of the end effector from the first angular position of the end effector.

14. An articulation system for a surgical tool, comprising:
a proximal universal joint including a proximal end member and a distal end member;
a hollow elongated member having a first end, a second end, and a longitudinal axis, the first end of the elongated member first end being mounted to the distal end member of the proximal universal joint;
an end effector universal joint comprising a proximal end member and a distal end member, the proximal end member of the end effector universal joint being mounted to the second end of the elongated member; and
universal joint control cables operatively connecting the proximal universal joint and the end effector universal joint,
wherein pivoting motion of the proximal end member of the proximal universal joint relative to the longitudinal axis of the elongated member exerts force on the control cables to cause a corresponding pivoting motion of the distal end member of the end effector universal joint.

15. The articulation system of claim 14, further comprising
a base control element rigidly mounted to the proximal end member of the proximal universal joint, and
a cable tensioner pivotally mounted to the base control element,
wherein the control cables are operatively connected to the tensioner.

16. The articulation system of claim 14, further comprising a brake assembly for engaging the proximal joint.

17. The articulation system of claim 14, further comprising a detensioner including
a base member operatively connected to the first end of the elongated member,
a slide member operatively mounted to the base member for linear movement relative to the base member between a first position adjacent the base member and a second position spaced from the base member, the slide member engaging the proximal joint, and
an arm pivotally connected to the base member for rotation in a plane parallel to the direction of linear movement of the slide member, and
means for biasing the slide member to the first position,
wherein rotation of the arm engages and moves the slide member from the first position to the second position.

18. The articulation system of claim 14, wherein each end member of the proximal universal joint and the end effector universal joint includes a base portion and opposing arms extending from the base portion, wherein each respective proximal end member and distal end member are mounted to a center member at the arms of the proximal end member and the distal end member, and wherein the center members permit pivoting of the proximal and distal end members around two substantially coplanar, perpendicular axes through the center member.

19. The articulation system of claim 18, wherein the proximal universal joint and the end effector universal joint each include round elements interposed between the center member and the arms at the mounting locations of the end members to the center member, and which may be independent parts or integral to the center member or arms, and wherein the round elements are engaged by the universal joint control cables.

20. The articulation system of claim 18, wherein four universal joint control cables operatively connect the proximal universal joint and the end effector universal joint.

21. The articulation system of claim 14, wherein the proximal end member of the proximal universal joint having a longitudinal axis and a first angular position, and the distal end member of the end effector has a longitudinal axis and a first angular position, and
wherein at all relative positions of the proximal end member of the proximal universal joint and the distal end member of the end effector universal joint, the longitudinal axis of the proximal end member of the proximal universal joint and the longitudinal axis of the distal end member of the end effector remain parallel, and the degree of rotation of the proximal end member of the proximal universal joint about the longitudinal axis of the proximal end member of the proximal universal joint from the first angular position of the proximal end member of the proximal universal joint is equal to the degree of rotation of the distal end member of the end effector universal joint about the longitudinal axis of the distal end member of the end effector from the first angular position of the end effector universal joint.
